(19) United States
(12) Patent Application Publication
(10) Pub. No.: US 2026/0015410 A1
(43) Pub. Date: Jan. 15, 2026

(54) HIGHLY FUCOSYLATED RECOMBINANT HUMAN ALPHA 1 ANTITRYPSIN (AAT) PROTEIN HAVING IMMUNOMODULATORY ACTIVITY AND COMPOSITIONS COMPRISING THE SAME

(71) Applicant: KAMADA LTD., Rehovot (IL)

(72) Inventors: Ayelet GRINHUT, Kibbutz Palmachim (IL); Danielle SHEVES, Modiin (IL); Michal AYALON, Tel Aviv (IL); Boris SCHWARTSBURD, Rehovot (IL)

(21) Appl. No.: 19/112,312

(22) PCT Filed: Sep. 26, 2023
(86) PCT No.: PCT/IL2023/051033

§ 371 Date: Mar. 17, 2025

Related U.S. Application Data
(60) Provisional application No. 63/413,665, filed on Oct. 6, 2022.

(51) Int. Cl. C07K 14/81; A61K 38/00; A61P 9/10

(52) U.S. Cl. CPC C07K 14/8125; A61P 9/10; A61K 38/00

(57) ABSTRACT

Provided herein are recombinant AAT proteins having high fucosylation levels and enhanced immunomodulatory biological activity as compared to a plasma derived AAT. Further provided are compositions including the same and methods for preparation thereof.

### FIELD OF THE INVENTION

The present disclosure is directed to recombinant human AAT protein, having high fucosylation levels, compositions comprising the same and methods for preparation thereof. Further provided are methods of using the recombinant human AAT protein as an immunomodulator in subjects in need thereof.

### BACKGROUND OF THE INVENTION

Endogenous human α1-antitrypsin (AAT) is an abundant acute phase glycoprotein, which is about 4 fold elevated in the blood in response to acute inflammation. Hepatocytes are the major source of AAT, but it is also expressed in mononuclear phagocytes and neutrophils, megakaryocytes, islet cells, and intestinal epithelial cells. Plasmatic AAT is a 52 kDa single-chain protein synthetized as a 418 amino acid precursor. The loss of the 24-aa signal peptide creates a 394 amino acid mature protein containing three complex carbohydrate side chains linked by an N-glycosidic bond to three asparagine residues (Asn46, Asn83, and Asn247). As a protease inhibitor, the primary role of AAT is maintaining the protease/antiprotease balance in the lung protecting tissues from the enzymes of inflammatory cells, especially human neutrophil elastase (HNE) released by neutrophils during an inflammatory state. Besides its high specificity for HNE, AAT has also the ability to neutralize several other proteases including cathepsin G, proteinase 3, metalloproteases, cysteine and aspartic proteases. Normal plasma concentration of alpha-1 antitrypsin ranges up to 3.5 mg/ml. Under certain conditions, AAT diffuses into tissue spaces and forms a 1:1 complex with target proteases. The enzyme/inhibitor complex is then removed from circulation by binding to serpin-enzyme complex receptor and catabolized by the liver and spleen. [1] AAT deficiency (AATD) is a genetic disorder characterized by early-onset severe emphysema. In AATD patients, therapy with plasma-derived AAT (pdAAT), “augmentation therapy”, provides clinical improvement but requires weekly intravenous infusions.

Ischemia followed by reperfusion (IR) is a pathological condition characterized by an initial restriction of blood supply (oxygen) to an organ followed by the subsequent restoration of perfusion and concomitant reoxygenation. [2] Occlusion of the arterial blood supply results in a severe imbalance of metabolic supply and demand, causing tissue hypoxia. Subsequent restoration of blood flow and reoxygenation is associated with an exacerbation of tissue injury and a profound inflammatory response. [3] Ischemic injury is associated with systemic inflammation due to cytokine production and increased expression of adhesion molecules by hypoxic parenchymal and endothelial cells [4].

Many pathological processes contribute to IR associated tissue injury. Hypoxia is associated with impaired endothelial cell barrier function due to decreases in adenylate cyclase activity and intracellular cAMP levels and a concomitant increase in vascular permeability and leakage.

The ischemic period is associated with significant alterations in the transcriptional control of gene expression For example, ischemia is associated with an inhibition of oxygen-sensing prolylhydroxylase (PHD) enzymes because they require oxygen as a cofactor. Hypoxia-associated inhibition of PHD enzymes leads to the post-translational activation of hypoxia and inflammatory signaling cascades, which control the stability of the transcription factors hypoxia-inducible factor (HIF) and nuclear factor-κB (NF-κB), respectively. Reperfusion injury is characterized by autoimmune responses, including natural antibody recognition of neoantigens and subsequent activation of the complement system. Activation of innate and adaptive immune responses also occurs and contributes to injury, including activation of pattern-recognition receptors such as TLRs and inflammatory cell trafficking into the diseased organ [3].

In addition, ischemia and reperfusion leads to the activation of cell death programs, including apoptosis (nuclear fragmentation, plasma membrane blebbing, cell shrinkage and loss of mitochondrial membrane potential and integrity), autophagy-associated cell death (cytoplasmic vacuolization, loss of organelles and accumulation of vacuoles with membrane whorls) and necrosis (progressive cell and organelle swelling, plasma membrane rupture and leakage of proteases and lysosomes into the extracellular compartment). [2, 3]. IR elicited tissue injury contributes to morbidity and mortality in a wide range of pathologies, including myocardial infarction (MI), ischemic stroke, acute kidney injury, trauma, circulatory arrest and others. Ischemia-reperfusion injury is also a major challenge during organ transplantation and cardiothoracic, vascular and general surgery.[3] Tissue injury and cell death by necrotic, necroptotic, pyroptotic, apoptotic, and autophagic mechanisms, occur as a result of the initial ischemic insult and then subsequent damage induced by reperfusion [5]. Moreover, Exposure of a single organ to ischemia and reperfusion may subsequently cause inflammatory activation in other organs, eventually leading to multiorgan failure.[3] Acute myocardial infarction (AMI) is a leading cause of morbidity and mortality worldwide [6]. Following AMI, the ischemic damage, and the reperfusion injury associated with restoration of blood flow, promote cell death, initiate an inflammatory response, and induce cardiac dysfunction eventually leading to heart failure [7]. Preclinical and clinical evidence suggests that plasma-derived AAT has some beneficial effects in the treatment of acute MI, possessing cytoprotective and anti-inflammatory properties [8, 9].

Intestinal ischemia is a common (over 1 in 1000 hospital admissions) and life-threatening condition. In-hospital mortality ranges from 67% to 80%, mainly because of 1) delayed diagnosis due to the lack of specific biomarkers; 2) major risk of sepsis; and 3) lack of validated treatment. Indeed, intestinal ischemia is often diagnosed when the bowel is necrotic, necessitating resection and compromising survival [10]. Reperfusion causes massive ROS formation which activates molecular (chemokines/cytokines/coagulation cascade) and cellular (neutrophils/macrophages/lymphocytes/platelets) components of innate immunity, leading to local and systemic inflammation and cell death. As a consequence, IRI can disrupt the mucosal barrier, causing bacterial (endotoxin) translocation and sepsis [11].

Studies in animals and humans have shown that prolonged ischemia followed by reperfusion will harm the paracellular barrier of the epithelial lining, facilitating translocation of bacterial products such as endotoxins. [12] Endotoxins, such as lipopolysaccharides (LPS), bind to Toll-like receptor-4 (TLR-4), activate various immune responses and cause the release of inflammatory cytokines [13].

A single layer of tightly linked epithelial cells forms a barrier between the intestinal lumen and underlying innate immune cells in the lamina propria. This epithelial lining is continuously exposed to environmental factors and bacteria. In recent years, the intestinal epithelial cell is increasingly recognized as an important mediator of inflammation [14]. Thereby, the epithelium actively contributes to antimicrobial host defense and the maintenance of mucosal homeostasis.

Key pro-inflammatory mediators such as IL-1β, IL-6, IL-8 and TNFα are enhanced by serine proteases and hence blocked by serine protease inhibitors, in particular by AAT [15]. Furthermore, AAT induces the production and release of anti-inflammatory mediators such as IL-10 and IL-1-receptor antagonist (IL-1Ra) [16].

Glycosylation plays an important role in determining the immune modulatory properties of AAT. It has been shown that alterations that occur to AAT glycans at times of inflammation and in malignant conditions may have an impact on the functions of the protein. Fucosylation is a type of glycosylation, which in many cases considered as non-beneficial, and hence is removed, or in other cases used only as a biomarker.

Recombinant forms of human AAT have been described, with an intended use for the treatment of AAT deficiency emphysema [6]. For example, U.S. Pat. No. 8,357,661 is directed to Recombinant human Alpha1-antitrypsin. For example, Marie-Eve Lalonde et al., is directed to the production of α2,6-sialylated and non-fucosylated recombinant alpha-1-antitrypsin in CHO cells. For example, Izel Koyuturk et al., discloses High-level production of wild-type and oxidation-resistant recombinant alpha-1-antitrypsin in glycoengineered CHO cells. For example, Mary Ann Comunale et al., is directed to Linkage Specific Fucosylation of Alpha-1-Antitrypsin in Liver Cirrhosis and Cancer Patients: Implications for a Biomarker of Hepatocellular Carcinoma. For example, Ogawa, K., et al. is directed to Tri-antennary tri-sialylated mono-fucosylated glycan of alpha-1 antitrypsin as a non-invasive biomarker for non-alcoholic steatohepatitis: a novel glycobiomarker for non-alcoholic steatohepatitis. For example, Cormac McCarthy et al., is directed to the Role and Importance of Glycosylation of Acute Phase Proteins with Focus on Alpha-1 Antitrypsin in Acute and Chronic Inflammatory Conditions. For example, Cormac McCarthy et al., is directed to Increased Outer Arm and Core Fucose Residues on the N-Glycans of Mutated Alpha-1 Antitrypsin Protein from Alpha-1 Antitrypsin Deficient Individuals. For example, Xiaojuan Zhang et al., is directed to Alpha-1 antitrypsin as a novel biomarker and potential therapeutic approach for metabolic diseases.

Accordingly, there is a need in the art for improved recombinant human AAT compositions, which are safe, efficient, cost effective to produce and which exhibit enhanced beneficial biological activity as compared to plasma derived AAT, with minimized side effects.

### SUMMARY OF THE INVENTION

According to some embodiments, there is provided herein a recombinant human α1-antitrypsin (rAAT) exhibiting high fucosylation levels and enhanced immunomodulatory effect in target tissues, as compared to plasma derived AAT (pdAAT). Further provided herein are compositions including the advantageous recombinant AAT, methods for its preparation and purification from producing cells as well as methods of using the same for inducing immunomodulatory effects and for treating various inflammatory-related conditions. Such immunomodulatory effects include such effects as, but not limited to reduction in inflammation (anti-inflammatory activity), increasing of anti-inflammatory cytokines, cell damage reduction, tissue protection, wound healing, and the like, or any combination thereof.

According to some embodiments, the herein disclosed advantageous rAAT includes the full-length amino acid sequence as that of a human wild type (WT) protein, yet surprisingly it exhibits highly improved immunomodulatory effect as compared to a corresponding plasma-derived (i.e., native protein) human AAT, having the same amino acid sequence.

According to some embodiments, the advantageous rAAT disclosed herein, which is produced in Chinese Hamster Ovary (CHO) cells, exhibit very high levels of Fucosylated N-linked glycans, yet surprisingly, such high levels (over at least 75%) of fucosylation results in enhanced biological activity of the rAAT, as an immunomodulator, as further exemplified herein below, both under in-vitro and in-vivo settings.

Thus, according to some embodiments, surprisingly, although the rAAT disclosed herein is shown to be active as a neutrophil elastase inhibitor, its immunomodulatory activity exceeded that of plasma derived AAT. Interestingly, as detailed herein, the final clones that were selected after screening of immunomodulatory potency, exhibit very high levels of fucosylation on the N-linked glycans as compared to other, less efficient clones.

According to some embodiments, without wishing to be bound to any theory or mechanism, surprisingly, high levels of fucosylation of the recombinant AAT, at least partially contribute to the improved and enhanced immunomodulation and anti-inflammatory effect of the recombinant AAT, including tissue protection effects, as compared to a plasma derived AAT protein, which exhibits only low levels of fucosylation (about 10-fold less fucosylated N-glycans).

According to some embodiments, there is thus provided a recombinant human alpha1-antitrypsin (rhAAT), comprising at least about 80% fucosylated N-linked glycans.

According to some embodiments, the rhAAT includes at least about 85%, at least about 90%, at least about 93% fucosylated glycans.

According to some embodiments, over about 70%, over about 80%, over about 85%, or over about 90% of the fucose units are core fucose.

According to some embodiments, about 60-80% of the fucosylated N-linked glycans are Di-antennary. According to some embodiments, about 70% of the fucosylated N-linked glycans are Di-antennary.

According to some embodiments, about 5-20% of the fucosylated N-linked glycans are Tri-antennary. According to some embodiments, about 10% of the fucosylated N-linked glycans are Tri-antennary.

According to some embodiments, about 5-20% of the fucosylated N-linked glycans are Tetra-antennary. According to some embodiments, about 10% of the fucosylated N-linked glycans are Tetra-antennary.

According to some embodiments, the rhAAT exhibits an increased immunomodulation activity as compared to the activity of a purified plasma derived AAT (pdAAT).

According to some embodiments, the rhAAT exhibit at least about 5% more, at least about 10% more, at least about 20%, at least about 50% more immunomodulation activity as compared to the activity of a purified plasma derived AAT.

According to some embodiments, the immunomodulation activity may be characterized in reduction in activity, expression and/or secretion level of one or more of: IL-1 beta, INF alpha, IL-6, IL-8, IL-18 and MCP1/CCL2.

According to some embodiments, the immunomodulation activity may be characterized in increasing activity, expression and/or secretion level of IL-10 and/or IL-1-receptor antagonist (IL-1Ra).

According to some embodiments, the rhAAT may be produced or expressed in Chinese hamster ovary (CHO) cell line. In some embodiments, the CHO cell line is CHO DG44 cell line. In some embodiments, the CHO cell line may further express \acute{α}-2,6-sialyltransferases. In some embodiments, the \acute{α}-2,6-sialyltransferases is exogenously expressed in the cells.

According to some embodiments, there is provided a pharmaceutical composition which includes the rhAAT and a pharmaceutically acceptable carrier.

According to some embodiments, the pharmaceutical composition is for use in treating or preventing an inflammatory condition in a subject in need thereof.

According to some embodiments, there is provided a method for preventing or treating an inflammatory condition in a subject in need thereof, the method includes administering a therapeutically effective amount of the rhAAT of the pharmaceutical composition including the same.

According to some embodiments, the inflammatory condition is a pulmonary disease selected from the group consisting of alpha-1 antitrypsin deficiency (AATD), small airway disease, chronic bronchitis, emphysema, chronic obstructive pulmonary disease (COPD), cystic fibrosis, bronchiectasis, asthma, pneumonia, parenchymatic and fibrotic lung diseases or disorders, interstitial pulmonary fibrosis, re-inflammation, acute respiratory distress syndrome (ARDS), and sarcoidosis.

According to some embodiments, the inflammatory condition may be selected from graft-versus-host disease (GVHD), ischemia-reperfusion injury, ischemia/reperfusion injury following transplantation, acute myocardial infarction, acute kidney injury, rheumatoid arthritis, septic arthritis, psoriatic arthritis, ankylosing spondylitis, Wegener's disease, Crohn's disease, ulcerative colitis, psoriasis, type I diabetes, dermatitis, pneumonia, sepsis, wound healing, and systemic lupus erythematosus. Each possibility is a separate embodiment. According to some embodiments, the inflammatory condition is ischemia-reperfusion injury.

According to some embodiments, rhAAT or the pharmaceutical composition may be administered at a dose of from about 1 mg/kg to about 500 mg/kg. In some embodiments the rhAAT or the pharmaceutical composition may be administered at a dose of from about 200 mg/kg to about 350 mg/kg, when administered by injection. In some embodiments, the rhAAT or the pharmaceutical composition may be administered at a dose of from about 20 mg/dose-200 mg/dose when administered by inhalation.

According to some embodiments, the administration may be performed once, 1-3 times per day, 1-7 times per week, 1-4 times per months, or any combination thereof.

According to some embodiments, the rhAAT or the pharmaceutical composition may be administered by injection.

According to some embodiments, the rhAAT or the pharmaceutical composition may be administered by inhalation.

According to some embodiments, there is a provided a method for producing rhAAT having high fucosylation levels, the method includes the steps of diluting and filtering the cell harvest; performing a first step of chromatography (for example, using POROS XQ Column); performing conductivity adjustment (for example, by dilution and Concentration by Ultra filtration (UF)); performing a second Chromatography (for example, using Capto Adhere Column); and exchanging buffer (for example, by Ultrafiltration/Diafiltration) to obtain a purified rAAT protein. The method may further optionally include a step of bulk filling and storage at suitable conditions (for example, Storage at −80° C.).

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1—shows an illustration of a plasmid map of the rhAAT expression vector. CMV-Cytomegalovirus promoter, DHFR—Dihydrofolate reductase;

FIG. 2—shows a Flow chart of the rAAT purification process, according to some embodiments;

FIGS. 3A-D—show representative results of purification from lab scale production runs:

FIG. 3A—shows POROS XQ chromatogram; FIG. 3C shows Capto™ Adhere chromatograms. Correlated pictograms of SDS-PAGE analysis are shown in FIG. 3B and FIG. 3D, respectively. rAAT peak identified in the chromatograms is marked by a frame, and the corresponding lane on the SDS-PAGE gel is marked by an arrow. M-marker, S/St—standard, H—harvest, L—load, U—unbound, W—wash, E—elution, R—regeneration, F—flow through;

FIG. 4—shows a representative chromatogram of final fraction with the monomer peak area percent shown in the inserted table;

FIG. 5A—Bar graphs showing effect on LPS-induced KC secretion by various concentrations of recombinant AAT clones (rhAAT clone 26 and clone 36) and Plasma derived AAT (pdAAT) in Splenocytes. The cells were treated with different concentration of AAT, Vehicle and Dexamethasone. KC secretion was determined by ELISA and presented as percentage of reduction compared to the vehicle control. The inhibition of cytokine secretion by rAAT was calculated by comparing cytokine concentration in the presence or absence (control) of AAT. Results represent means±SEM of wells in each group. *p<0.05, **p<0.01, ***p<0.001 statistics are presented for each AAT treatment compared to vehicle+LPS treated cells, according to t-Test;

FIG. 5B—Bar graphs showing effect on LPS-induced TNF secretion by various concentrations of recombinant AAT clones (rhAAT clone 26 and clone 36) vs. pdAAT in Splenocytes. The cells were treated with different concentration of AAT, Vehicle and Dexamethasone. TNFa secretion was determined by ELISA and presented as percentage of reduction compared to the vehicle control. The inhibition of cytokine secretion by rAAT was calculated by comparing cytokine concentration in the presence or absence (control) of AAT. Results represent means±SEM of wells in each group. *p<0.05, **p<0.01, ***p<0.001 statistics are presented for each AAT treatment compared to vehicle+LPS treated cells, according to t-Test;

FIG. 6A—Bar graphs showing effect on LPS-induced TNF secretion by various concentrations of recombinant AAT vs. pdAAT in murine macrophages. TNFa secretion was determined by ELISA and presented as percentage of reduction compared to the vehicle control. The inhibition of cytokine secretion by rAAT was calculated by comparing cytokine concentration in the presence or absence (control) of AAT. Results represent means±SEM of wells in each group. *p<0.05, **p<0.01, ***p<0.001 statistics are presented for each AAT treatment compared to vehicle+LPS treated cells, according to t-Test;

FIG. 6B—Bar graphs showing effect on LPS-induced CXCL1 secretion by various concentrations of recombinant AAT vs. pdAAT in murine macrophages. CXCL1 secretion was determined by ELISA and presented as percentage of reduction compared to the vehicle control. The inhibition of cytokine secretion by rAAT was calculated by comparing cytokine concentration in the presence or absence (control) of AAT. Results represent means±SEM of wells in each group. *p<0.05, **p<0.01, ***p<0.001 statistics are presented for each AAT treatment compared to vehicle+LPS treated cells, according to t-Test;

FIG. 6C—Bar graphs showing effect on LPS-induced IL-10 secretion by various concentrations of recombinant AAT vs. pdAAT in murine macrophages. IL-10 secretion was determined by ELISA and presented as percentage of increase compared to the vehicle control. IL-10 secretion was determined by ELISA and presented as percentage of increase compared to the vehicle control. Results represent means±SEM of wells in each group. *p<0.05, **p<0.01, ***p<0.001 statistics are presented for each treatment compared to vehicle+LPS treated cells, according to t-Test;

FIG. 7A—Bar graphs showing effect on LPS-induced IL-6 secretion by various concentrations of two recombinant AAT clones (C26 and C36) vs. pdAAT in PBMC cells. Freshly isolated human PBMCs were treated with different concentration of AAT, Vehicle and Dexamethasone. IL-6 secretion was determined by ELISA and presented as percentage of reduction compared to the vehicle control. Results represent means±SEM of wells in each group. *p<0.05, **p<0.01, ***p<0.001 statistics are presented for each treatment compared to vehicle+LPS treated cells, according to t-Test;

FIG. 7B—Bar graphs showing effect on LPS-induced TNF secretion by various concentrations of two recombinant AAT clones vs. pdAAT in PBMC cells. Freshly isolated human PBMCs were treated with different concentration of AAT, Vehicle and Dexamethasone. TNFa secretion was determined by ELISA and presented as percentage of reduction compared to the vehicle control. Results represent means±SEM of wells in each group. *p<0.05, **p<0.01, ***p<0.001 statistics are presented for each treatment compared to vehicle+LPS treated cells, according to t-Test;

FIG. 7C—Bar graphs showing effect on LPS-induced IL-10 secretion by various concentrations of two recombinant AAT clones vs. pdAAT in PBMC cells. Freshly isolated human PBMCs were treated with different concentration of AAT, Vehicle and Dexamethasone. IL-10 secretion was determined by ELISA and presented as percentage of reduction compared to the vehicle control. Results represent means±SEM of wells in each group. *p<0.05, **p<0.01, ***p<0.001 statistics are presented for each treatment compared to vehicle+LPS treated cells, according to t-Test;

FIGS. 8A-C—Bar graphs showing the effect of rAAT on left ventricular (LV) systolic function in myocardial ischemia reperfusion (IR) injury in mice. CD1 mice (8-10 weeks old) underwent myocardial IR by transient left coronary artery occlusion for 30 minutes. rAAT or control were given via intraperitoneal injection immediately after initiation of reperfusion. Control sham surgery were performed without occluding the coronary artery. After 24 hours of reperfusion the mice were anesthetized with isoflurane (1.5-3%) and echocardiography was performed to measure left ventricular fractional shortening (LVFS) in order to calculate the left ventricular ejection fraction (LVEF) (FIG. 8A). Infarct size under different conditions are shown in FIG. 8B (% area at risk) and FIG. 8C (infracted LV segments). For measurements of infract size triphenyl tetrazolium chloride (TTC) was used. The hearts were removed and perfused with normal saline solution containing 2.5 mM CaCl₂. Subsequently, 1% Evans blue dye was injected into the aorta and the heart was then perfused again with normal saline solution to wash out the excess Evans blue. Finally, the heart was frozen, and cut into slices that were then incubated in a 1% TTC isotonic phosphate buffer at room temperature for 30 min. The areas of infarcted tissue, the area at risk, and the whole LV were determined using ImageJ software. Results represent means±SEM; and

FIGS. 9A-H—show the effect of rAAT on wound healing in tissue culture cells. The tissue protective activity of rAAT was assessed in wound healing (gap repair by cell migration) in A549 cells or human umbilical vein endothelial cells (HUVEC). Gaps were made by straight scratch in 30 degrees angle, simulating a wound in confluent cell layer. Cells were treated with 1 mg/ml of pdAAT and two clones of rAAT in 2% serum medium. Control was treated by vehicle (saline). FIGS. 9A-F show pictograms at TO and after 24 hours (T24) after treatment; FIGS. 9A-9B—control treatment (saline); FIGS. 9C-9D—rAAT (clone 26); FIGS. 9E-9F—rAAT (clone 36); FIG. 9G shows bar graphs of percentage of wound closure on A549 cells, as calculated by ImageJ software, after 16 hours and after 24 hours of treatment with control, pdAAT, rAAT (clone 26) and rAAT (clone 36). Results are presented as means±SEM.

FIG. 9H shows the effect of rAAT on wound healing in HUVEC. Gaps were made by Ibidi culture-inserts. Cells were treated with 1 mg/ml of pdAAT, rAAT clone 26 or saline control in 5% serum medium. FIG. 9H shows bar graph of percentage of wound colure as calculated by ImageJ software, after 13 hours and after 18 hours of treatment with control, pdAAT and rAAT (clone 26). Results are presented as means±SEM.

### DETAILED DESCRIPTION OF THE INVENTION

According to some embodiments, there is provided herein is a recombinant human AAT having full length wild type sequence with high fucosylation levels (at least over 75%), that can be produced in large quantities in CHO cells. Such recombinant AAT is exemplified herein to possess elastase inhibition activity similar to pdAAT while exhibiting highly superior immunomodulatory activity.

According to some embodiments, native, plasma derived AAT includes three glycosylation sites, however, as exemplified herein, it is surprisingly shown that at least 75%, at least 80%, at least 85% or at least 90% of the N-linked glycans of the herein disclosed recombinant human AAT, are fucosylated.

According to some embodiments, as exemplified herein, the immunomodulatory activity of the recombinant AAT was assessed in cell-based assays using primary culture from C57BL/6 male mice spleen and human peripheral blood mononuclear cells (PBMC), as well as in-vivo in Acute myocardial infarction (IMI) model.

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As used herein, the term “AAT” and “AAT-1” may interchangeably be used. The terms are directed to human alpha (α)1 anti-trypsin. In some embodiments, the AAT has a nucleotide sequence as denoted by SEQ ID NO: 1 and an amino acid sequence as denoted by SEQ ID NO: 2.

The terms “plasma derived AAT” and “pdAAT” are interchangeable, and are directed to an AAT that has been derived/purified/produced from human plasma. In some embodiments, pdAAT is a native protein.

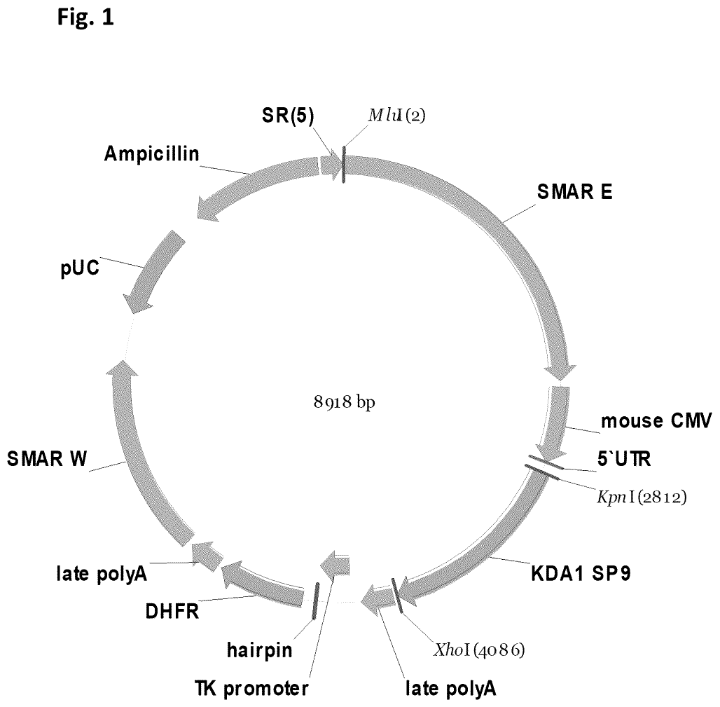

The terms “recombinant AAT”, “human recombinant AAT”, “rAAT”, “hrAAT” and “rhAAT” are interchangeable, and are directed to a recombinant AAT that has been expressed from an exogenous expression plasmid introduced to producing cells. A schematic illustration of an exemplary plasmid for expressing a recombinant AAT is shown in FIG. 1, according to some embodiments. In some embodiments, the cells are Chines Hamster Ovary (CHO) cells. In some embodiments, the CHO cells are DG44. In some embodiments, the CHO cells further optionally express \acute{α}-2,6-sialyltransferases.

As used herein, the term “treating” includes, but is not limited to one or more of the following: abrogating, ameliorating, inhibiting, attenuating, blocking, suppressing, reducing, delaying, halting, alleviating or preventing symptoms associated with a condition. Each possibility represents a separate embodiment. In some embodiments, the condition is an immune related condition, such as, an inflammatory condition.

As used herein, N-linked glycans are sugar chains that are covalently linked to asparagine residues of a polypeptide. The N-linked glycans can branch, yielding two (referred to herein as “Di” or “di”), three (referred to herein as “Tri” or “tri”) or four (referred to herein as “Tetra” or “tetra”) antennas.

Fucosylation is a type of glycosylation, which includes adding fucose sugar units to the polysaccharides. Fucose is a hexose deoxy sugar, lacking an hydroxyl group on carbon 6 (C-6) and having an L-configuration. In fucosylated N-linked glycans (i.e., fucose-containing glycan structures), fucose can exist as structure-specific fucosylation—“core fucosylation” or as “antennary fucosylation”.

Native AAT protein has three glycosylation sites. Thus, AAT includes three N-linked glycans in which branching can occur. Surprisingly, it has been shown herein that the recombinant AAT disclosed herein has a high degree of fucosylation, in particular, core fucosylation, in which fucose is linked to the innermost N-glycan residue.

Thus, according to some embodiments, there is provided a recombinant human AAT protein having a degree of fucosylation in the range of about 70-95%, or subranges thereof. In some embodiments, the degree/level of fucosylation is about 80%. In some embodiments, the degree/level of fucosylation is about 85%. In some embodiments, the degree/level of fucosylation is about 90%. In some embodiments, the degree/level of fucosylation is about 93%. In some embodiments, the degree/level of fucosylation is at least about 75%. In some embodiments, the degree/level of fucosylation in at least about 80%. In some embodiments, the degree/level of fucosylation is at least about 85%. In some embodiments, the degree/level of fucosylation is at least about 88%. In some embodiments, the degree/level of fucosylation is at least about 90%. In some embodiments, the degree/level of fucosylation is at least about 91%. In some embodiments, the degree/level of fucosylation is at least about 92%. In some embodiments, the degree/level of fucosylation is at least about 93%. In some embodiments, the degree of fucosylation is over about 75%, over about 80%, over about 85%, or over about 90%.

According to some embodiments, about 50-99% of the fucose units are core fucose units. In some embodiments, over about 50% of the fucose units are core units. In some embodiments, over about 60% of the fucose units are core units. In some embodiments, over about 70% of the fucose units are core units. In some embodiments, over about 80% of the fucose units are core units. In some embodiments, over about 85% of the fucose units are core units. In some embodiments, over about 90% of the fucose units are core units.

According to some embodiments, about 55-80% of the fucosylated N-linked glycans are Di-antennary. In some embodiments, at least about 55% of the fucosylated N-linked glycans are Di-antennary. In some embodiments, at least about 60% of the fucosylated N-linked glycans are Di-antennary. In some embodiments, at least about 65% of the fucosylated N-linked glycans are Di-antennary. In some embodiments, at least about 70% of the fucosylated N-linked glycans are Di-antennary. In some embodiments, at least about 75% of the fucosylated N-linked glycans are Di-antennary

According to some embodiments, about 5-25% of the fucosylated N-linked glycans are Tri-antennary. According to some embodiments, at least about 5% of the fucosylated N-linked glycans are Tri-antennary. According to some embodiments, at least about 7.5% of the fucosylated N-linked glycans are Tri-antennary. According to some embodiments, at least about 10% of the fucosylated N-linked glycans are Tri-antennary. According to some embodiments, at least about 15% of the fucosylated N-linked glycans are Tri-antennary. According to some embodiments, at least about 20% of the fucosylated N-linked glycans are Tri-antennary.

According to some embodiments, about 5-20% of the fucosylated N-linked glycans are Tetra-antennary. According to some embodiments, at least about 5% of the fucosylated N-linked glycans are Tetra-antennary. According to some embodiments, at least about 7.5% of the fucosylated N-linked glycans are Tetra-antennary. According to some embodiments, at least about 8.5% of the fucosylated N-linked glycans are Tetra-antennary. According to some embodiments, at least about 10% of the fucosylated N-linked glycans are Tetra-antennary.

According to some embodiments, the fucosylation levels of the rAAT are at least 2-12 times higher as compared to the fucosylation level of plasma derived AAT (native AAT). In some embodiments, the fucosylation levels of the rAAT are at least 2 times higher as compared to the fucosylation level of pdATT. In some embodiments, the fucosylation levels of the rAAT are at least 3 times higher as compared to the fucosylation level of pdATT. In some embodiments, the fucosylation levels of the rAAT are at least 4 times higher as compared to the fucosylation level of pdATT. In some embodiments, the fucosylation levels of the rAAT are at least 5 times higher as compared to the fucosylation level of pdATT. In some embodiments, the fucosylation levels of the rAAT are at least 6 times higher as compared to the fucosylation level of pdATT. In some embodiments, the fucosylation levels of the rAAT are at least 7 times higher as compared to the fucosylation level of pdATT. In some embodiments, the fucosylation levels of the rAAT are at least 8 times higher as compared to the fucosylation level of pdATT. In some embodiments, the fucosylation levels of the rAAT are at least 9 times higher as compared to the fucosylation level of pdATT. In some embodiments, the fucosylation levels of the rAAT are at least 10 times higher as compared to the fucosylation level of pdATT. In some embodiments, the fucosylation levels of the rAAT are at least 11 times higher as compared to the fucosylation level of pdATT. In some embodiments, the fucosylation levels of the rAAT are at least 12 times higher as compared to the fucosylation level of pdATT.

According to some embodiments, in order to determine N-glycosylation level, in particular, fucosylation levels of a protein, various analytical methods can be used, including, for example, Liquid Chromatography-Mass Spectrometry (LC-MS) analysis. An LC-MS analysis of AAT proteins (recombinant and plasma derived) is exemplified hereinbelow (Example 4).

According to some embodiments, the rAAT disclosed herein may be produced in CHO cells. In some embodiments, the CHO cells are CHO DG44 cell line. Such cell lines may be introduced (for example, by transfection) under suitable conditions, such as, a suitable medium (for example, serum free medium), with a plasmid/expression vector (such as the expression plasmid illustrated in FIG. 1), containing the nucleotide sequence encoding the open reading frame (ORF) of human alpha 1 anti-trypsin (Represented by SEQ ID NO: 1), under the control of a promoter. In order to identify AAT expressing clones, selection makers, such as, DHFR and Neomicin may be used. In some embodiments, the cells may further optionally be engineered to express \acute{α}-2,6-sialyltransferases, for example, by transfection of alpha-2,6 sialyltransferase coding sequence. In some embodiments, the AAT and the alpha-2,6 sialyltransferase may be con-transfected into the CHO cells, in separate or common expression vector. In some embodiments, each gene (i.e., AAT and alpha-2,6 sialyltransferase) may be under the control of a separate or similar promoter.

According to some embodiments, there is thus provided a process for the production of rAAT in CHO cells, the process includes culturing suitable CHO host cells under conditions allowing the expression of the rAAT polypeptide from a vector introduced into the cells, and optionally recovering/isolating the produced polypeptide from the cell culture.

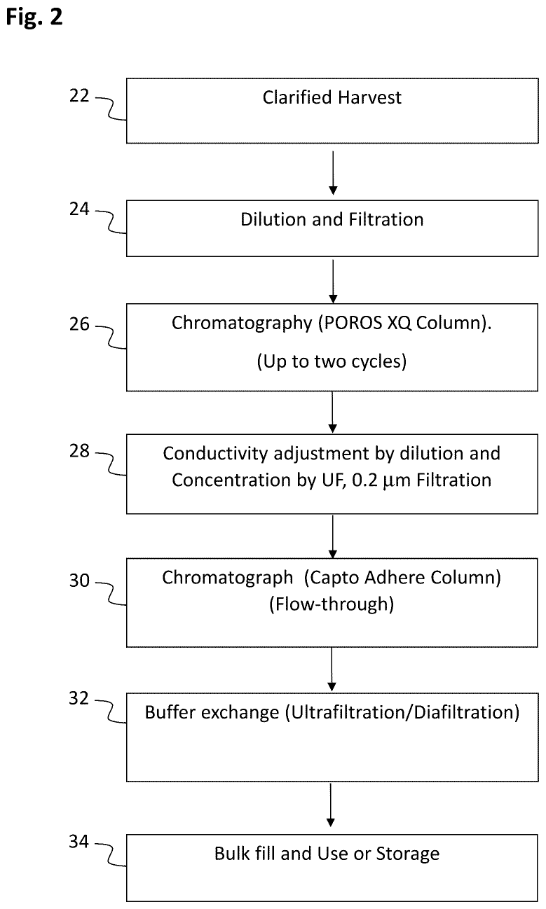

According to some embodiments, there is provided a method for recovering rATT from CHO cells, while maintaining the integrity of the protein and preserving its activity. Reference is made to FIG. 2, which schematically illustrate a method for recovering AAT from CHO cells, the method includes at step 22 obtaining clarified CHO cells harvest expressing rAAT. At step 24, the harvest is diluted and filtered. The dilution may be at any ration, such as, for example, 1:3 dilution with a suitable buffer, and the filtration may include, for example, filtration of 0.2 μm. At step 26, the samples of step 24 are processed by chromatography, using, for example, POROS XQ Column. The chromatography separation may be repeated for any number of cycles, for example, 1-3 cycles. At step 28, The conductivity of the samples is adjusted by dilution with a suitable buffer, and further concentrated by utilizing ultrafiltration (UF) process. At step 30, the sample are re-processed by chromatography, using, for example, Capto Adhere Column, which is utilized in a flow-through mode. At step 32, buffer exchanging of the samples, is performed, using, for example, Ultrafiltration/Diafiltration system, with a cut-off of 10 kDa and 0.2 μm. Next, at final step 34, the samples are transferred to suitable receptacles (such as, vials, containers, etc.) for use or for storage, for example at 4° C., −20° C. or −80° C.

According to some embodiments, by the methods disclosed herein, clones of rATT having high fucosylation levels and enhanced immunomodulatory activity have been identified. Such clones are also termed herein as “C-26” and “C-36”.

According to some embodiments, the rAAT obtained from the cells may be used as is, or may be used in a pharmaceutical composition, in the presence of one or more suitable excipients. The suitable excipients may be selected according to the purpose, type and/or use of the composition. In some embodiments, excipient is a pharmaceutical excipient which may include or a pharmaceutical carrier, vehicle, buffer and/or diluent.

According to some embodiments, the rAAT of the composition disclosed herein may be used as an immunomodulator and as medicament for treating various immune related conditions.

According to some embodiments, the immunomodulation activity of the rAAT may be characterized in reduction in the activity, expression and/or secretion level of one or more of: IL-1 beta, TNF alpha, IL-6, IL8, IL 18, MCP1/CCL2.

According to some embodiments, the immunomodulation activity may be characterized in increasing the activity or expression level of IL-10 and IL-1-receptor antagonist (IL-1Ra).

According to some embodiments, as exemplified herein, surprisingly, the rAAT exhibit enhanced (increased) immunomodulation activity as compared to a plasma derived AAT, under similar conditions, despite having the same amino acid sequence.

In some embodiments, the rAAT exhibit an increased immunomodulation activity as compared to a plasma derived (native) AAT (pdAAT). In some embodiments, the increased immunomodulation activity is about 2-2500% higher. In some embodiments, the rAAT exhibits at least about 2% higher immunomodulation activity compared to pdAAT. In some embodiments, the rAAT exhibits at least about 5% higher immunomodulation activity compared to pdAAT. In some embodiments, the rAAT exhibits at least about 10% higher immunomodulation activity compared to pdAAT. In some embodiments, the rAAT exhibits at least about 20% higher immunomodulation activity compared to pdAAT. In some embodiments, the rAAT exhibits at least about 30% higher immunomodulation activity compared to pdAAT. In some embodiments, the rAAT exhibits at least about 40% higher immunomodulation activity compared to pdAAT. In some embodiments, the rAAT exhibits at least about 50% higher immunomodulation activity compared to pdAAT. In some embodiments, the rAAT exhibits at least about 60% higher immunomodulation activity compared to pdAAT. In some embodiments, the rAAT exhibits at least about 70% higher immunomodulation activity compared to pdAAT. In some embodiments, the rAAT exhibits at least about 80% higher immunomodulation activity compared to pdAAT. In some embodiments, the rAAT exhibits at least about 90% higher immunomodulation activity compared to pdAAT. In some embodiments, the rAAT exhibits at least about 100% higher immunomodulation activity compared to pdAAT. In some embodiments, the rAAT exhibits at least about 150% higher immunomodulation activity compared to pdAAT. In some embodiments, the rAAT exhibits at least about 200% higher immunomodulation activity compared to pdAAT. In some embodiments, the rAAT exhibits at least about 300% higher immunomodulation activity compared to pdAAT. In some embodiments, the rAAT exhibits at least about 400% higher immunomodulation activity compared to pdAAT. In some embodiments, the rAAT exhibits at least about 500% higher immunomodulation activity compared to pdAAT. In some embodiments, the rAAT exhibits at least about 1000% higher immunomodulation activity compared to pdAAT. In some embodiments, the rAAT exhibits at least about 2000% higher immunomodulation activity compared to pdAAT. In some embodiments, the rAAT exhibits at least about 2500% higher immunomodulation activity compared to pdAAT. In some embodiments, the increased immunomodulation activity is about 1.5-50 fold higher compared to the activity of pdAAT. In some embodiments, the increased immunomodulation activity is about 5-40 fold higher compared to the activity of pdAAT. In some embodiments, the increased immunomodulation activity is about 10-30 fold higher compared to the activity of pdAAT. In some embodiments, the increased immunomodulation activity is about 16-20 fold higher compared to the activity of pdAAT. In some embodiments, the increased immunomodulation activity is about 1.5 fold higher compared to the activity of pdAAT. In some embodiments, the increased immunomodulation activity is about 2 fold higher compared to the activity of pdAAT. In some embodiments, the increased immunomodulation activity is about 3 fold higher compared to the activity of pdAAT. In some embodiments, the increased immunomodulation activity is about 4 fold higher compared to the activity of pdAAT. In some embodiments, the increased immunomodulation activity is about 5 fold higher compared to the activity of pdAAT.

According to some embodiments, the rAAT or the composition including the same may be used for treating an inflammatory condition in a subject in need thereof. In some embodiments, the inflammatory condition is a lung disease or conditions. In some embodiments, the lung disease or condition may be selected from, but not limited to: alpha-1 antitrypsin deficiency (AATD), small airway disease, chronic bronchitis, emphysema, chronic obstructive pulmonary disease (COPD), cystic fibrosis, bronchiectasis, asthma, pneumonia, parenchymatic and fibrotic lung diseases or disorders, interstitial pulmonary fibrosis, re-inflammation, acute respiratory distress syndrome (ARDS) and sarcoidosis. Each possibility is a separate embodiment.

In some embodiments, the inflammatory condition may be selected from, but not limited to: graft-versus-host disease (GVHD), ischemia-reperfusion injury, ischemia/reperfusion injury following transplantation, myocardial infarction, Acute kidney injury (AKI), rheumatoid arthritis, septic arthritis, psoriatic arthritis, ankylosing spondylitis, Wegener's disease, Crohn's disease, ulcerative colitis, psoriasis, type I diabetes, dermatitis, pneumonia, sepsis, wound healing, systemic lupus erythematosus, and multiple sclerosis. Each possibility is a separate embodiment.

According to some embodiments, the rAAT or the composition including the same may be administered by any suitable route of administration, that may be determined in accordance with the treated condition, type of tissue, subject characteristics, and the like.

According to some embodiments, administration routes may include local and systemic routes. Exemplary suitable routes of administration include, but are not limited to: orally, intra-nasally, parenterally, intravenously, topically, enema or by inhalation. According to another embodiment, systemic administration is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to another embodiment, parenteral administration is transmucosal administration. According to another embodiment, transmucosal administration is transnasal administration. For transmucosal administration, penetrants appropriate to the barrier to be permeated are used in the formulation. Such penetrants are generally known in the art. The preferred mode of administration will depend upon the particular indication being treated and will be apparent to one of skill in the art.

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides.

According to some embodiments, the rAAT or composition is administered intravenously, and is thus formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

According to another embodiment, administration systemically is through an enteral route. According to another embodiment, administration through an enteral route is buccal administration. According to another embodiment, administration through an enteral route is oral administration. According to some embodiments, the composition is formulated for oral administration.

According to some embodiments, oral administration is in the form of hard or soft gelatin capsules, pills, capsules, tablets, including coated tablets, dragees, elixirs, suspensions, liquids, gels, slurries, syrups or inhalations and controlled release forms thereof.

According to some embodiments, the administration may include any suitable administration regime, depending, inter alia, on the medical condition, patient characteristics, administration route, and the like. In some embodiments, administration may include a one-time administration. In some embodiments, administration may include administration once a day, twice daily, every day, every other day, every third day, every fourth day, every fifth day, once a week, once every second week, once every third week, once every month, and the like. In some embodiments, the administration may include administration of a loading dose and maintenance with increasing intervals, such as, for example, administering a loading dose on day 0, maintenance on days 1, 3, 5, 7 and then weekly or biweekly chronic administration. In some embodiments, administration may include administration 1-3 times per day, for 1-7 days per week, for 1-4 times per month.

According to some embodiments, the administration may include any suitable dosing regime, depending, inter alia, on the medical condition, patient characteristics, administration route, and the like. In some embodiments, dosing by parenteral route (such as, injection), may be in an amount ranging from about 1 mg/kg to about 500 mg/kg. In some embodiments, the dosing may be in an amount ranging from about 1.5 mg/kg to about 450 mg/kg. In some embodiments, the dosing may be in an amount ranging from about 5 mg/kg to about 400 mg/kg. In some embodiments, the dosing may be in an amount ranging from about 10 mg/kg to about 350 mg/kg. In some embodiments, the dosing may be in an amount ranging from about 20 mg/kg to about 300 mg/kg. In some embodiments, the dosing may be in an amount ranging from about 50 mg/kg to about 250 mg/kg. In some embodiments, the dosing may be in an amount ranging from about 100 mg/kg to about 200 mg/kg. In some embodiments, dosing by inhalation route, may be in an amount ranging from about 10 to about 300 mg/dose. In some embodiments, dosing by inhalation route, may be in an amount ranging from about 20 to about 200 mg/dose.

According to some embodiments, the rAAT or the composition including the same, when used for used for treating an immune-related condition/inflammatory condition may be used in combination with other therapeutic agents. The components of such combinations may be administered sequentially or simultaneously/concomitantly in separate or combined pharmaceutical formulations by any suitable administration route.

According to some embodiments, there is provided a method of treating an immune related condition, such as, an inflammatory condition, the method includes administration to a subject in need thereof a therapeutically effective amount of rATT, or a composition comprising the same. In some embodiments, the rAAT may be administered as a polypeptide as is, or in a suitable pharmaceutical composition.

According to some embodiments, a therapeutically effective amount refers to an amount sufficient to ameliorate and/or prevent at least one of the symptoms associated with the immune-related disorder or inflammatory condition.

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1—Generation of rAAT Producing Cells

CHO DG44 cells were transfected under serum free condition, with a plasmid containing the nucleotide sequence encoding the open reading frame (ORF) of human alpha 1 anti-trypsin (Represented by SEQ ID NO: 1, which also includes a nucleotide sequence encoding a signal peptide (underlined)). The sequence was codon optimized to the hamster codon usage. AAT gene was expressed by the strong promotor of cytomegalovirus (CMV). The selection markers were DHFR and Neomicin. The plasmid map is presented in FIG. 1.

The cells were further optionally engineered to express \acute{α}-2,6-sialyltransferases by transfection with hamster beta-galactosyl alpha-2,6 sialyltransferase (ST6Gal1). The alpha-2,6 sialyltransferase may be under the control of a separate promoter.

After the first transfection, eight pools were subjected to single cell cloning procedure using FACS for single cell separation and ultrahigh-throughput imaging system for monoclonality verification. 4000 clones were isolated and propagated in 384, 96, 24, 12 and 6 well plates and later were subjected to growth in shake flask. A step wise selection was made during the propagations and was based on AAT titer as determined by ELISA (AAT EISA kit, ICL) and the presence of sialic acid capping by lectin-based assay. Final top 48 clones which were proven to be monoclonal with the best volumetric productivity, specific productivity (PCD) and growth parameters were selected for final assessment in a bioreactors system (Sartorious GmbH).

SEQ ID NO: 1 (human alpha 1 anti-trypsin, including signal peptide sequence
(underlined))
(SEQ ID NO: 1)
atggatctgctgcacaagaacatgaagcacctgtggttctttctgctgctggtggccgctcctagatgggtgttgtctgaaga
tcctcagggcgacgccgctcagaaaaccgatacctctcaccacgaccaggatcaccccaccttcaacaagatcaccccta
acctggccgagttcgccttcagcctgtacagacagctggcccaccagtccaactctaccaacatcttcttcagccccgtgtct
atcgccaccgcctttgctatgctgtctctgggcaccaaggctgacacccacgatgagatcctggaaggcctgaacttcaac
ctgacagagatccccgaggctcagatccacgagggctttcaagagctgctgagaaccctgaaccagcctgactctcagct
ccagctgacaaccggcaacggcctgtttctgtctgagggcctgaagctggtggacaagttcctggaagatgtgaagaagct
gtaccactccgaggccttcaccgtgaacttcggcgataccgaggaagccaagaagcagatcaacgactacgtggaaaag
ggcacccagggcaagatcgtggacctggtcaaagagctggacagagacaccgtgttcgccctggtcaactacatctttttc
aaaggcaagtgggaacgccccttcgaagtgaaggacacagaggaagaggacttccacgtcgaccaagtgaccaccgtg
aaggtgcccatgatgaagcggctgggcatgttcaacatccagcactgcaagaaactgtcctcttgggtgctgctgatgaagt
acctgggcaacgctaccgctatcttcttcctgcctgatgagggcaagctgcagcacctggaaaacgagctgacccacgac
atcatcaccaagttcttggagaacgaggaccggcggagcgcttctctgcatctgcctaagctgtctatcaccggcacctacg
acctgaagtctgtgctgggacagctgggcatcacaaaggtgttctctaacggcgccgatctgtccggcgtgacagaagaa
gctcctctgaagctgtccaaggccgtgcataaggctgtgctgaccatcgatgagaagggaacagaggccgctggcgcca
tgtttctggaagctatccctatgagcatccctcctgaagtgaagttcaacaagcccttcgtgttcctgatgatcgagcagaaca
ccaagtctccactgttcatgggcaaagtggtcaaccccacacagaagtgatag
Amino acid sequence of rAAT (including a signal peptide (underlined)) (SEQ ID NO: 2)
(SEQ ID NO: 2)
MDLLHKNMKHLWFFLLLVAAPRWVLSEDPQGDAAQKTDTSHHDQDHPTFN
KITPNLAEFAFSLYRQLAHQSNSTNIFFSPVSIATAFAMLSLGTKADTHDEILEG
LNFNLTEIPEAQIHEGFQELLRTLNQPDSQLQLTTGNGLFLSEGLKLVDKFLED
VKKLYHSEAFTVNFGDTEEAKKQINDYVEKGTQGKIVDLVKELDRDTVFALV
NYIFFKGKWERPFEVKDTEEEDFHVDQVTTVKVPMMKRLGMFNIQHCKKLS
SWVLLMKYLGNATAIFFLPDEGKLQHLENELTHDIITKFLENEDRRSASLHLP
KLSITGTYDLKSVLGQLGITKVFSNGADLSGVTEEAPLKLSKAVHKAVLTIDE
KGTEAAGAMFLEAIPMSIPPEVKFNKPFVFLMIEQNTKSPLFMGKVVNPTQK:

### Example 2—Purification of rAAT

A three step purification process was utilized to purify rAAT. Recombinant AAT was purified from 5 litres of cell culture harvest of AAT producing cell clones using a multi-step downstream process at lab scale. The purification steps were designed to meet the requirements of purity, yield and quality necessary for preclinical evaluation. The purification process is described below and depicted in FIG. 2.

The culture supernatant was diluted with 20 mM Tris HCl buffer till conductivity of 6 mS/cm was reached and filtered through a 0.2 μm filter. The harvest was subsequently captured on a POROS XQ column. After a washing step, the bound rAAT was eluted with 20 mM Tris-HCl, 0.4 M NaCl pH 7.4±0.1. The pooled eluted fractions were diluted with Tris-HCl based buffer (20 mM Tris-HCl, pH 7.2) to a conductivity level of 20 mS/cm and concentrated using 10 kDa UF membranes. The concentrated material was filtered by 0.2 μm filtration and then loaded on multimodal strong anion exchanger column Capto™ Adhere. These columns run in one cycle of flow-through (non-binding) mode. Flow-through collected material was filtered through 0.2 μm. The filtered material was concentrated and diafiltrated by UF/DF (10 kD) against pre-formulation solution, 150 mM NaCl. The final pre-formulation was filtered by 0.2 μm filter under aseptic conditions. Each 5-litre run resulted in 7-10 grams of purified AAT, representing yield of above 70%.

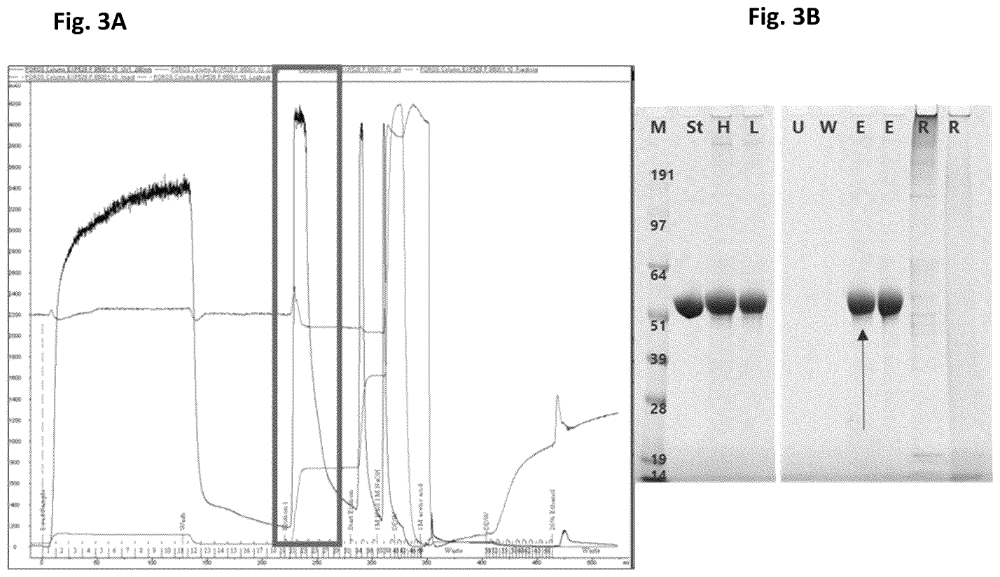

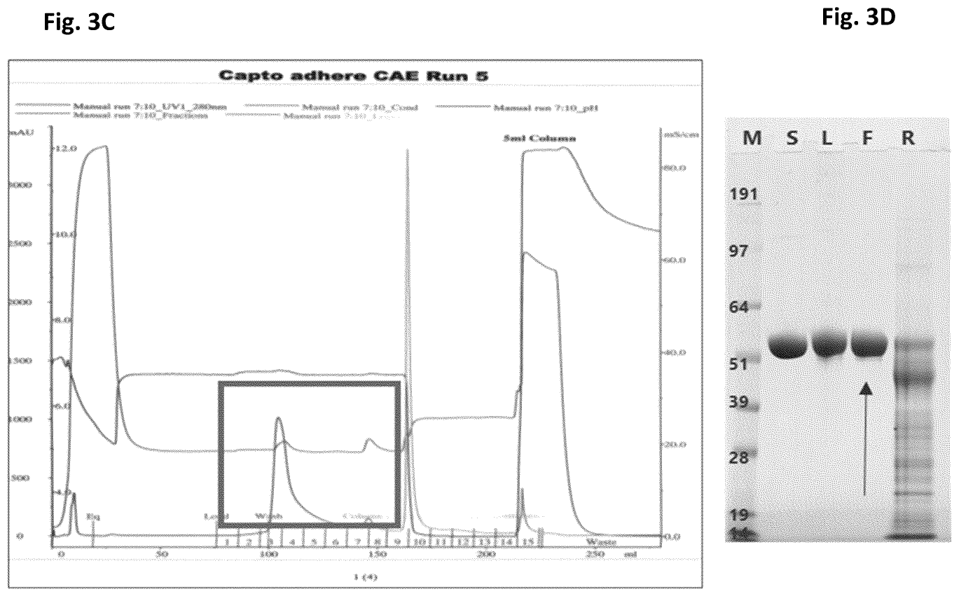

Typical results of purification from the lab scale production runs are presented in FIGS. 3A-D: FIG. 3A—shows POROS XQ chromatogram, and FIG. 3C shows Capto™ Adhere chromatograms. Correlated pictograms of the SDS-PAGE analysis are shown in FIG. 3B and FIG. 3D, respectively. rAAT peak identified in the chromatograms is marked by a frame, and the corresponding lane on the SDS-PAGE gel is marked by an arrow. M—marker, S/St—standard, H—harvest, L—load, U—unbound, W—wash, E—elution, R—regeneration, F—flow through.

### Example 3—Recombinant AAT Characterization

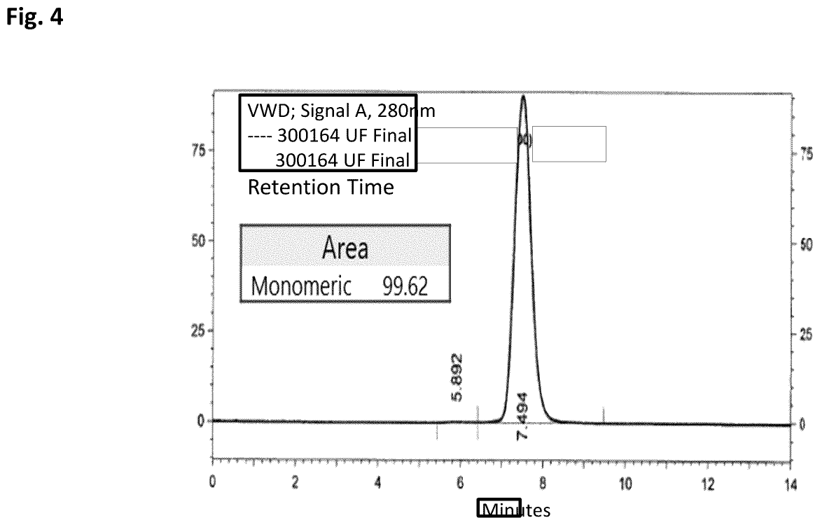

Antigenic AAT titer of starting material (cell culture harvest) was established by nephelometry and AAT ELISA. Purity was assessed by molecular size distribution (MSD) assay by SEC-TPLC. FIG. 4 shows a chromatogram of a final fraction with the monomer peak area percent of 99.2.

Table 1 below summarizes various properties of the rAAT.

TABLE 1

| Test | Results | |
|---|---|---|
| Appearance | Colorless to slightly yellow solution | |
| SDS-PAGE | Co-migrates with plasma derived AAT | |
| | standard (RHS#2) | |
| Isoform composition by IEF | Main 8 bands in pH range 4-5 | |
| Active AAT content by elastase | 62.3 | mg/ml |
| inhibition assay | | |
| Total protein concentration by UV | 72.5 | mg/ml |
| absorption at 280 nm (Abs_{0.1%} - 0.546) | | |
| AAT content by Immunonephelometry | 72.5 | mg/ml |
| AAT specific activity (mg/mL active | 86% | |
| rAAT/ mg/mL total protein OD280) | | |
| Purity by SDS-PAGE (densitometry) | 100% | |
| Purity by RP-HPLC (peak area %) | 98% | |
| Molecular size distribution, monomers | 97% | |
| (SEC-HPLC) | | |
| Total microbial count by Microbiological | 0 CFU/1 ml | |
| Examination USP <62> | | |
| pH by Potentiometry USP <791> | 7.4 | |
| Osmolality by Freezing point USP <785> | 286 | mOsmol/kg |

Table 2 below provides a summary of Characterization Results for rAAT by LC-MS peptide mapping.

TABLE 2

| Product | | |
|---|---|---|
| Attribute | Peptide mapping | Results |
| Primary | Sequence | The percentage of coverage of the amino acid |
| Structure | coverage | sequence was 96.2%. |
| | N-terminal | Peptide covering N-terminal: EDPQGDAAQK |
| | sequence | (SEQ ID NO: 3). The N-terminal amino acid |
| | | sequence was fully validated to demonstrate |
| | | correct cleavage of the signal peptide. |
| | C-terminal | Peptide covering C-terminal: |
| | peptide | FMGKVVNPTQK (SEQ ID NO: 4). The C- |
| | | terminal amino acid sequence confirmed. |
| Post- | N-terminal | Peptide covering N-terminal Glutamic acid (E): |
| Translational | Glutamic acid | EDPQGDAAQK (SEQ ID NO: 5). Most of the |
| Modifications | level evaluation | N-terminal glutamic acid remains intact and only |
| | | 0.4% is converted to the N-terminal pyroglutamic |
| | | acid. |
| | C-terminal | Peptide covering C-terminal Lysine (K): |
| | Lysine clipping | VVNPTQK (SEQ ID NO: 6). The clipped portion |
| | level evaluation | of Lys was found to be only 0.1%. |
| | Evaluation of free | Peptide covering Cysteine at position 232 (C): |
| | Cysteine 232 | RLGMFNIQHCK. (SEQ ID NO: 7)The free Cys |
| | level | 232 level is 45%. |
| | Evaluation of | Peptide covering Methionine at position 351 (M): |
| | Methionine 351 | AIPMSIPPEVK (SEQ ID NO: 8) |
| | and Methionine | Peptide covering Methionine at position 358 (M): |
| | 358 oxidation | PFVFLMIEQNTK (SEQ ID NO: 9). The level of |
| | level | oxidation of Methionine 351 and Methionine 358 |
| | | is relatively low and is in the range of 3% and |
| | | 8%, respectively. |
| | Evaluation of | No quantifiable deamidation observed for |
| | Asparagine and | Glutamine 40, 44, 97, 212 and 305, and for |
| | Glutamine | Asparagine 46, 49, 81, 83,146, 186, 247, 278 and |
| | deamidation level | 367. |

### Example 4—N-Glycan Profiling by LC-MS Analysis

For further analysis, Clone 26 (C-26) and clone 36 (C-36) were selected.

In order to assess N-Glycan profiling by LC-MS analysis, N-glycans from various protein samples (plasma derived AAT or rhAAT clones) were released and fluorescently labeled with the Waters Glycoworks RapiFluor-MS N-Glycan Kit (P/N 176003713) according to the manufacturer's instructions using 2-aminobenzamide (2-AB) as a fluorescent label.

An aliquot of each sample was diluted with PBS to a final concentration of 1 mg/mL. For each sample, a volume of 15 μL (equivalent to approximately 15 g) of diluted sample solution was prepared in an Eppendorf tube. Each sample prepared was subjected to denaturation by addition of 7.8 μL water and 6 μL of a 5% RapiGest™ solution (3 mg RapiGest SF Surfactant+60 μL 5× Glycoworks Rapid Buffer) and incubation for 3 min at 110° C. The denatured samples were cooled down for 3 min at room temperature. The N-glycans were released by addition of 1.2 μL of GlycoWorks Rapid PNGase F and incubation for 5 min at 57° C. The samples were let to cool down for 3 min at room temperature.

The released N-glycans were subjected to labelling with a fluorescent tag by addition of 12 μL of RapiFluor-MS labelling solution (9 mg GlycoWorks RapiFluor-MS Reagent Powder+131 μL GlycoWorks RapiFluor-MS Reagent Solvent) and incubation for 5 min at room temperature. Following reaction, the samples were diluted using 358 μL acetonitrile. The labelled N-glycans were cleaned-up by solid phase extraction using Waters Glycoworks HILIC μElution plate mounted over a positive pressure device. Wells were first conditioned with 200 μL H2O then with two times 200 μL of H2O/ACN (15/85). Each ACN diluted sample was loaded then washed twice with 600 μL of HCOOH/H2O/ACN (1/9/90). The N-glycans were finally eluted using three times 30 μL of Glycoworks SPE Elution buffer. The purified samples were diluted using 310 μL of GlycoWorks Sample Diluent (32/68 DMF/ACN) prior to LC-MS analysis.

Labelled N-glycans were analyzed on LC-MS. LC-MS System used is a Waters UPLC Integrated System Acquity UPLC I-Class with fluorescence detector coupled on-line to a Waters Xevo G2-S Q-ToF. Separation was performed on a BEH Glycan column 100 mm×2.1, 1.7 μm (Waters, Milford, MA, Cat. No.: 186004742) and Solvents: A: 100% Acetonitrile, B: 50 mM Ammonium Format, pH 4.4 adjusted with Formic acid and filtered 0.2 μm. Separation gradient from 39% buffer A to 47% buffer A over 16 min at 0.5 mL/min flow-rate. Fluorescence detector set to high power lamp and 360 nm excitation, 428 nm emission. MS settings: Acquisition was performed in positive electrospray MSE scanning mode over the m/z range 50-4000, processed RT range 10 to 30 min UPLC-MS data were processed using a validated UNIFI software version 1.8.2. The relative quantification of each glycoform detected was finally performed within the retained population of N-glycans detected above the 1% reporting threshold.

The results are presented in Table 3 below, showing the rAAT C26 and C36 clones N-glycan profiles compared to plasma derived AAT (pdAAT, Glassia), and more particularly, the percentage of fucosylated N-glycans in the various samples.

TABLE 3

| | | Source of AAT | | | |
|---|---|---|---|---|---|
| | | ^{a}rAAT expressed by | ^{b}rAAT expressed by | ^{c}pdAAT | ^{c}pdAAT |
| | | C26 CHO cell line | C36 CHO cell line | Glassia, RHS#2 | Glassia, RHS#3 |
| N-Link | | Relative Peak | Relative Peak | Relative Peak | Relative Peak |
| structure | Assignment | Response (%) | Response (%) | Response (%) | Response (%) |
| Di- | FA2 | 3.29 | 0.74 | <LOQ | <LOQ |
| antennary, | A2G1 | ^{d}n.d. | 0.36 | <LOQ | <LOQ |
| fucosylated | FA2G1 | 4.34 | 0.31 | <LOQ | <LOQ |
| and | A2G2 | 1.34 | 0.27 | <LOQ | <LOQ |
| afucosylated | FA2G2 | 13.74 | 1.37 | <LOQ | <LOQ |
| | FA2G1S1 | 26.11 | 8.26 | <LOQ | <LOQ |
| | A2G2S1 | 0.98 | 1.48 | <LOQ | <LOQ |
| | A2G2S2 | 2.62 | n.d. | 78.20 | 80.60 |
| | FA2G2S2 | 24.52 | 54.91 | 7.10 | 7.40 |
| Tri- | A3G3S1 | n.d. | 2.71 | <LOQ | <LOQ |
| antennary, | FA3G3S1 | n.d. | 4.91 | <LOQ | <LOQ |
| fucosylated | FA3G3S2 | 3.81 | 6.91 | <LOQ | <LOQ |
| and | A3G3S3 | n.d. | n.d | 14.50 | 12.00 |
| afucosylated | FA3G3S3 | 5.31 | 8.82 | n.d. | n.d. |
| Tetra- | FA4G4S3 | 5.80 | 3.68 | n.d. | n.d. |
| antennary, | FA4G4S4 | 6.52 | 4.22 | n.d. | n.d. |
| fucosylated | | | | | |
| and | | | | | |
| afucosylated | | | | | |
| Total | | 93.44 | 94.13 | 7.10 | 7.40 |
| amount of | | | | | |
| structures | | | | | |
| fucosylated | | | | | |

^{a}rAAT—recombinant AAT;
^{b}glycoengineered rAAT by co-expression with \acute{α}-2,6-sialyltransferase; [C36 clone was co-expressed with \acute{α}-2,6-sialyltransferases]
^{c}pdAAT—plasma derived AAT;
^{d}n.d.—not detected; LOQ—Limit of quantification.

Thus, the results presented above clearly demonstrate that the rATT proteins of the present disclosure (i.e., clones 26 and 36) have a very high degree of fucosylation (over 10 fold), as compared to a plasma-derived AAT protein.

### Example 5—Biological Activity of rAAT In-Vitro

To test the immunomodulatory effect of the rAAT of the present disclosure, various in-vitro studies were performed on mice splenocytes, murine macrophages and human peripheral blood mononuclear cells (PBMC). The test revealed the surprisingly enhanced effect of the rAAT on the level of various immune-related markers.

### I. Mouse Splenocytes CXCL1/KC, TNFα

Studies were performed to assess the efficacy of recombinant AAT clones compared to plasma derived AAT in inhibiting LPS-induced KC (CXCL1) and TNFα secretion in murine splenocytes. Splenocytes were freshly isolated from C57BL/6 male mouse spleen, resuspended and seeded into 96 well plate, 5*10⁵ cells/well. The cells were pre-incubated with different concentration of pdAAT and different clones of rAAT for 5 hours, replenished with fresh AAT at the same concentration and followed by stimulation with 10 ng/mL LPS (Sigma Cat #L2630) for additional 24 hours. Then, the cell supernatant from each well was collected and evaluated for KC concentration (CXCL1) by specific ELISA (R&D Cat #DY453) and TNFα concentration (R&D Cat #DY410).

Statistical evaluation of the data was performed using mixed linear models (LMM), a statistical model containing both fixed and random effects. Statistical analysis was performed on four final clones, as well as pdAAT. In all tested concentrations, clone 26 was significantly more potent than other clones and pdAAT, with an effect peaking at 0.5 mg/mL and reaching up to 93% reduction in KC secretion and 90% reduction in TNFα secretion.

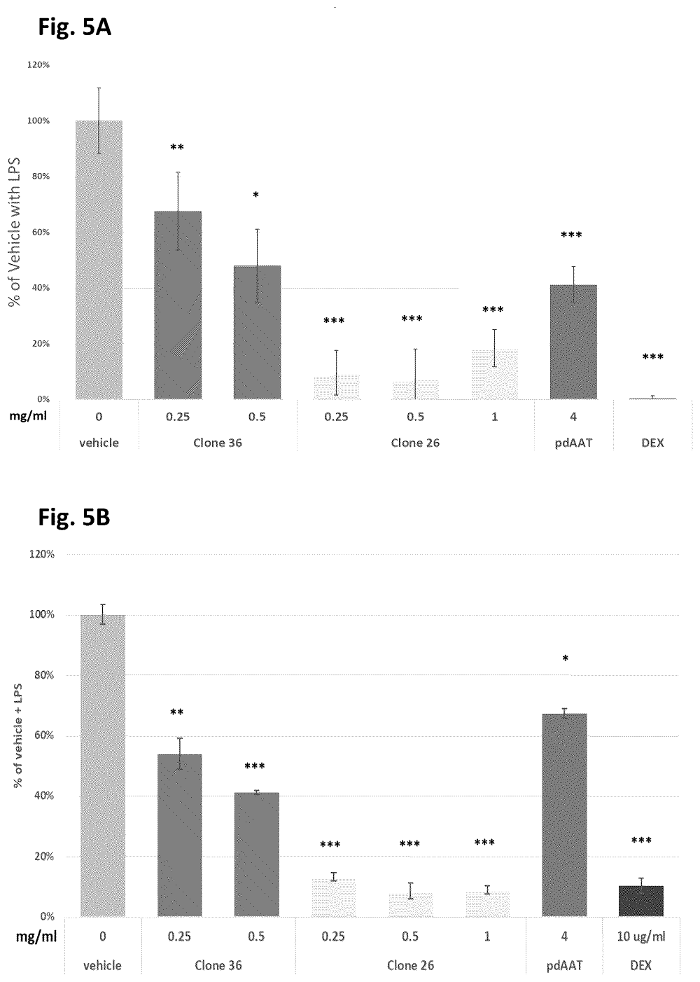

As shown in FIG. 5A, significant reduction of LPS induced KC secretion was identified at all tested concentrations: For rAAT clone 36 at 0.25-0.5 mg/mL, for rAAT clone 26 at 0.25-1 mg/mL and for pdAAT only at 4 mg/mL. The maximal effect on KC reduction was observed for rAAT clone 26 at 0.25-0.5 mg/mL between ˜82-93% reduction, with optimal reduction at 0.5 mg/ml.

As shown in FIG. 5B, significant reduction of LPS induced TNFα secretion was identified at all tested concentrations of both C26 and C36 clones but the highest reduction was observed with clone 26, at 0.25-1 mg/mL, showing reduction of between about 85-90%.

### II. RAW 264.7 Murine Macrophages Cell Line TNFa, CXCL1/KC, IL-10

The anti-inflammatory activity of recombinant AAT was also tested in murine macrophages. Cells were seeded into 96 well plate, 5*10⁵ cells/well. Cells were either treated with different concentration of pdAAT and rAAT and stimulated with 5 ng/ml LPS for 24 h, or pre-incubated with different concentration of pdAAT and rAAT for 5 hours, replenished with fresh AAT at the same concentration, followed by stimulation with 5 ng/mL LPS (Sigma Cat #L2630) for additional 24 hours. Then, the cell supernatant from each well was collected and evaluated for KC concentration (CXCL1) by specific ELISA (R&D Cat #DY453), TNFα concentration (R&D Cat #DY410) and IL-10 concentration (R&D cat #DY417).

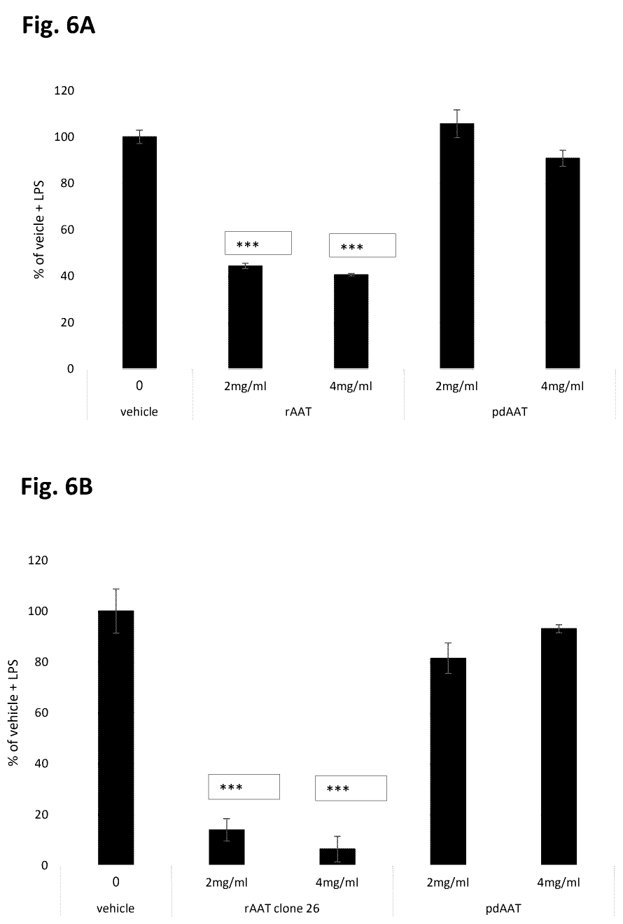

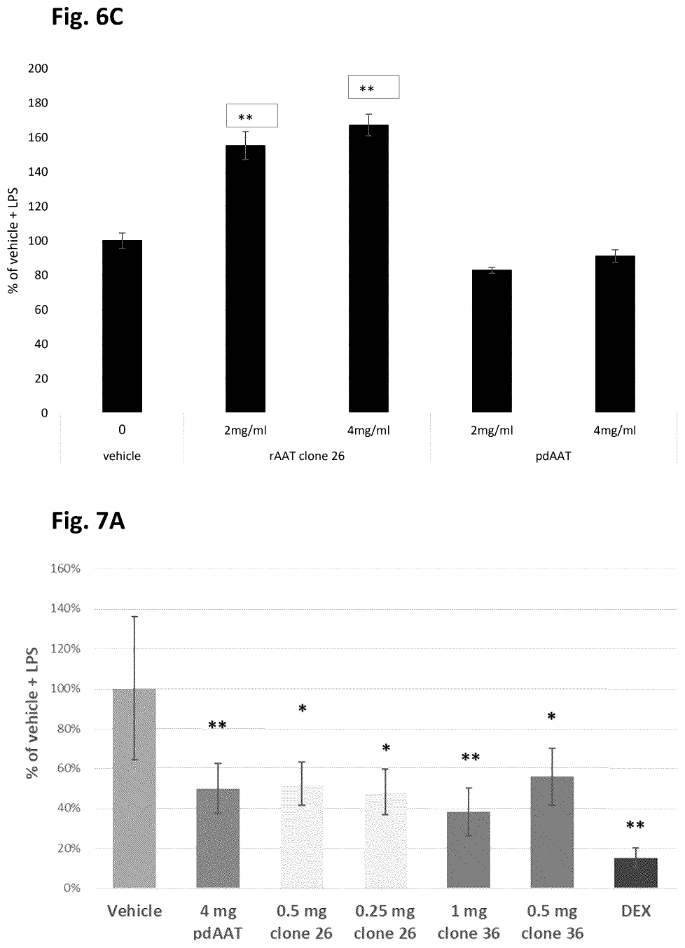

As shown in FIG. 6A, significant reduction of LPS induced TNFα secretion was identified in both tested concentrations of rAAT. rAAT was significantly more potent than pdAAT with a TNFa reduction effect of between 56-60%.

As shown in FIG. 6B, significant reduction of LPS induced CXCL1 secretion was identified in both tested concentrations of rAAT. rAAT was significantly more potent than pdAAT with a CXCL1 reduction effect of between 86-94%.

Interestingly, as shown in FIG. 6C, both concentrations of recombinant AAT increased anti-inflammatory IL-10 secretion by more than 50% while pdAAT did not affect its secretion.

### III. Human PBMC IL-8, IL-6, TNFa, IL-10

Human PBMCs were freshly prepared by leukapheresis separation from healthy donors. Cells were seeded into 96 well plate, 5*10⁵ cells/well. The cells were pre incubated with different concentration of pdAAT and rAAT for 5 hours, replenished with fresh AAT at the same concentration and followed by stimulation with 10 ng/mL LPS (Sigma Cat #L2630) for additional 24 hours. Then, the cell supernatant from each well was collected and concentrations of IL-8, IL-6, IL-10 and TNFα (TNFalpha/TNFa) were determined by specific ELISAs.

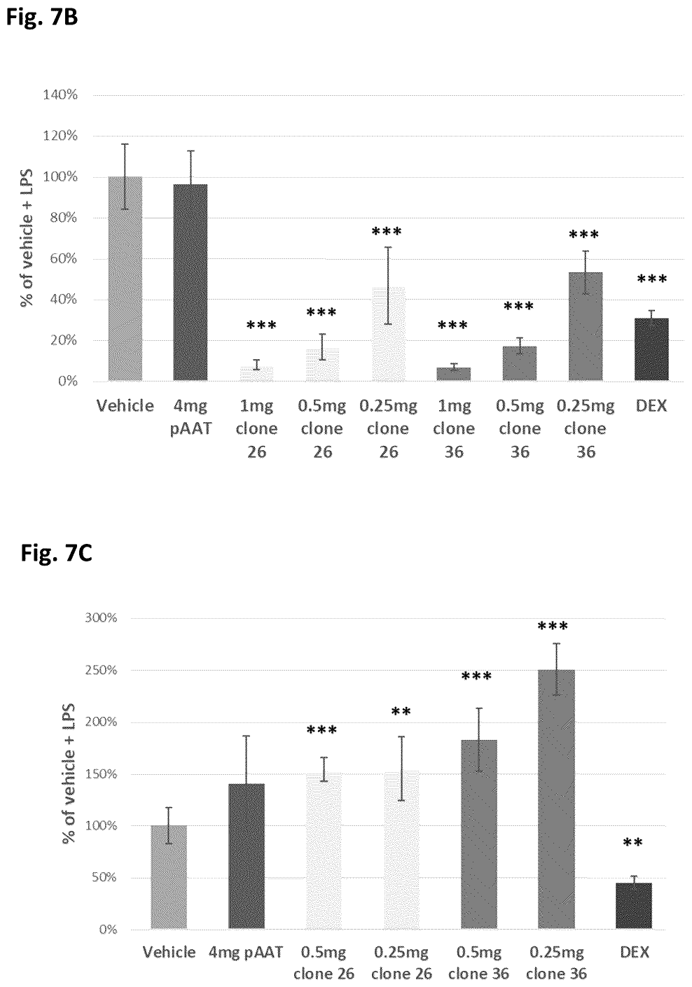

The results are presented in FIGS. 7A-C. As shown in FIG. 7A, both clones and pdAAT reduced the secretion of IL-6 by 40-55%. However, while 4 mg/ml of pdAAT was needed for 50% inhibition, only 1/16 (0.25 mg/ml) of clone 26 was needed to induce the same effect.

rAAT Clone 26 was the most potent also in inhibition of LPS-induced TNFα secretion in human PBMCs. As shown in FIG. 7B, 1 mg/ml of both recombinant clones inhibited more than 90% of TNFα secretion, while pdAAT showed no effect even when using a 4-fold higher concentration.

Interestingly, while Dexamathasone (a corticosteroid that prevents the release of cytokines that cause inflammation) inhibited also anti-inflammatory IL-10 secretion (as shown in FIG. 7C), plasma derived AAT and recombinant AAT increased its secretion in a dose dependent manner. pdAAT contribution was not significant at a concentration of 4 mg/ml but both recombinant clones increased IL-10 by more than 50%.

### Example 6—Biological Activity of rAAT In-Vivo

The biological activity of rAAT in vivo was evaluated in an acute myocardial infarction model (AMI) in mice. Infarct size and cardiac function using echocardiography were assessed as detailed below.

Adult male CD1 mice (8-10 weeks old) underwent experimental myocardial ischemia/reperfusion (IR) by transient left coronary artery occlusion for 30 minutes, followed by reperfusion for 24 hours. Mice were anesthetized using ketamine/xylazine/acepromazine (100/15/1 mg/kg) followed by orotracheal intubation and connection to a rodent ventilator (Minivent, Harvard Apparatus). After 30 minutes the artery occlusion was released to initiate the reperfusion. Following the verification of successful reperfusion, the rAAT (C26) or control treatment were given via intraperitoneal injection. Control sham surgery were performed without occluding the coronary artery.

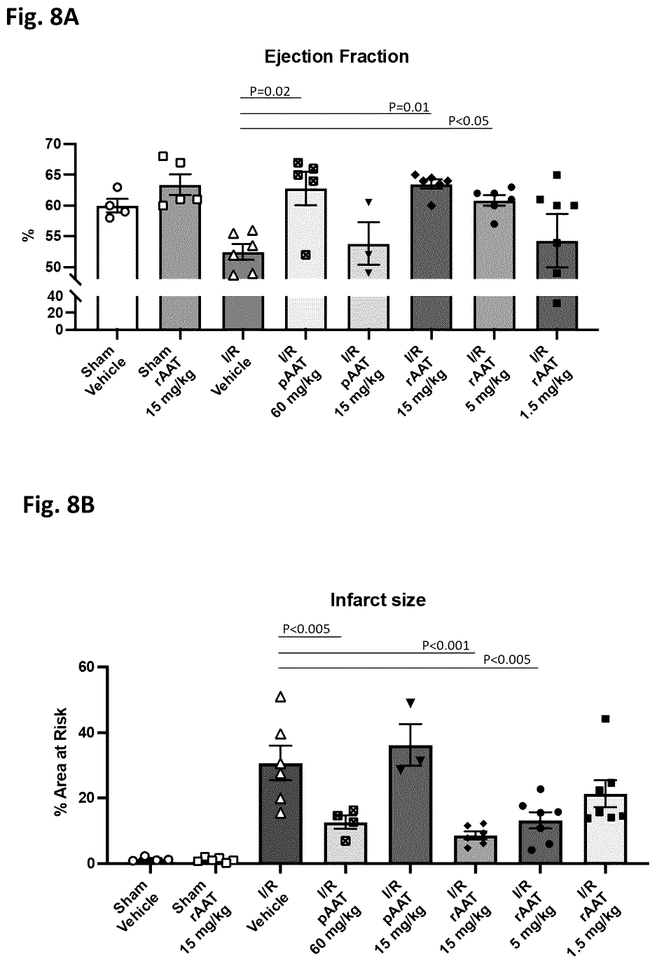

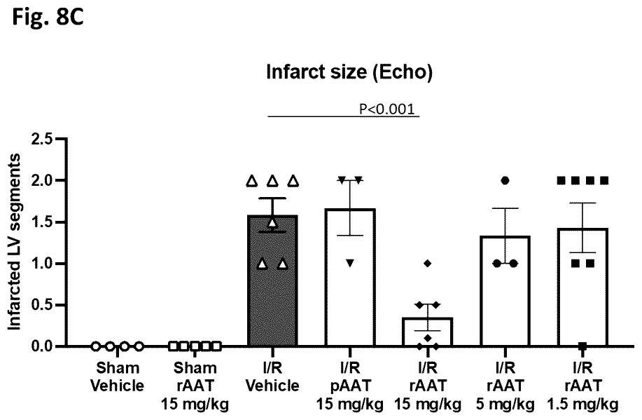

After 24 hours, the mice were anesthetized with isoflurane (1.5-3%) and echocardiography was performed to measure left ventricular fractional shortening (LVFS) in order to calculate the left ventricular ejection fraction (LVEF). The results are shown in FIG. 8A.

Myocardial damage was also determined by pathology assessment of viability using 10% triphenyl tetrazolium chloride (TTC, Sigma Aldrich) in PBS to stain the viable myocardium, and phthalo blue color (Heucotech) to mark the non-risk area, spared by the ischemic injury. Infarct size was expressed as a percentage of the whole LV myocardium (FIG. 8C) and as a percentage of the area-at-risk (AAR), as shown in FIG. 8B.

For additional assessment of myocardial damage, plasma obtained from the mice was used to measure cardiac troponin I (cTnI) using an ELISA assay (Life Diagnostics). ELISA was used to detect plasma IL-18 as a measurement of inflammasome activation.

The results clearly demonstrate that treatment of mice with rAAT clone 26 resulted in a significant reduction in infarct size. There was an approximate 60-70% reduction in infarct size in mice treated with rAAT clone 26 at 15 mg/kg dose, compared to vehicle treated mice. In mice treated with 5 mg/kg dose, 50% reduction was observed indicating a dose-response effect. In addition, treatment with 15 mg/kg dose of rAAT clone 26 led to a significant preservation in LV systolic function measured as LVEF at echocardiography after 24 hours from I/R injury and to an increase in injection fraction.

### Example 7—Biological Activity of rAAT In-Vivo in Intestinal Ischemia Reperfusion (I/R) Injury Model

Cell culture and animal studies have shown that AAT limits tissue injury and promotes cell and tissue survival. AAT can promote tolerance in animal models by downregulating early inflammation and favouring induction and stabilization of regulatory T cells. AAT was shown to reduce the intestinal permeability in murine models of inflammatory bowel disease [17]. AAT has been shown to improve the outcome in different animal models of organ IRI systems: kidney, liver, lung, heart, brain.[2, 18-19]. However, there is no data on the effect of AAT on intestinal IRI. As exemplified herein, the biological activity of rAAT in vivo is evaluated in an intestinal ischemia reperfusion model (IRI) in rats. Survival of the rats following IRI was assessed as detailed below.

The experiments were performed on a validated rat model of intestinal IRI in which the animals were exposed to 60 minutes of intestinal ischemia followed by 7 days reperfusion and monitoring of survival group. Experiments were be performed in male Sprague-Dawley rats (280-350 grams). The surgeries were conducted under anesthesia using intraperitoneal injections of Ketamine (100 mg/mL)/Xylazine (20 mg/mL) as follows:
1. Standard laparotomy with dissection and clamping of the superior mesenteric artery and ischemic evaluation by paleness of the small bowel and lack of pulsation in the mesenteric vessels.
2. Temporary closure of the abdomen using clips.
3. After 60 minutes of warm ischemia, the clamp was removed, 1 mL of warmed NaCl 0.9% was administered intraperitoneally (compensating for fluid loss) and the abdomen and skin were closed with sutures (Prolene 3.0 and 4.0 respectively) in the 7 days group.

In the treatment arm, AAT was be administered intravenously, during ischemia and before reperfusion. The controls/shams received an equivalent volume of NaCl 0.9% only. In the sham group, the identical procedure was performed except the occlusion of the SMA.

Buprenorphine (Vetergesic© 0.3 mg/mL) was administered, subcutaneously, on day 0, 1 and 2 postoperative in the survival group.

At various fixed time points after reperfusion (180 minutes, 24 hours and 7 days) the animal can be anesthetised again and euthanised by exsanguination, in order to collect blood and intestinal samples. Samples from the terminal ileum are collected and immediately mounted on an Ussing chamber at 37° C., in the sacrifice groups, to measure the permeability and functionality. Jejunal, ileal and rectal samples are taken for histopathological assessment (in 4% formaldehyde) and gene expression analysis (snap frozen in liquid N2).

### Example 8—Tissue Protective Activity of rAAT In-Vitro

The tissue protective activity of rAAT in vitro was assessed in wound healing (gap repair by cell migration) model in A549 lung carcinoma epithelial cells and human umbilical vein endothelial cells (HUVECs).

### 1. A549 Lung Carcinoma Epithelial Cells

Cells were seeded 60,000/well on 24 well plate in 500 μl medium.

Mitomycin C was used to inhibit proliferation and distinguish between proliferation and migration of the cells.

Gaps were made by straight scratch using a 200 μl pipette tip in 30 degrees angle, simulating a wound in confluent cell layer.

Cells were treated with 1 mg/ml of pdAAT or two clones of rAAT (C26 or C36) in 2% serum medium. Control was treated by vehicle (saline).

Pictures of the cells were taken at time 0, 16 and 24 hours. In addition, the wound closure was calculated by ImageJ and presented as percentage of wound closure after 16 and 24 hours.

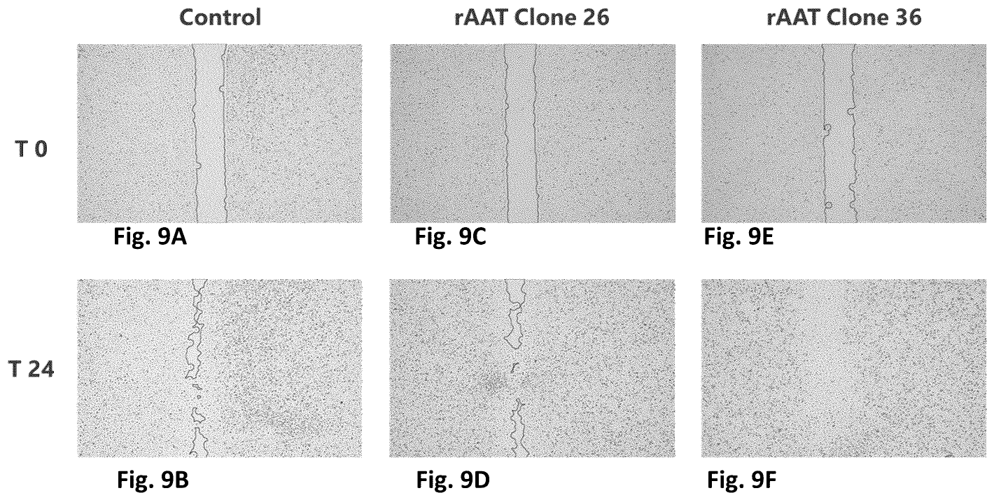

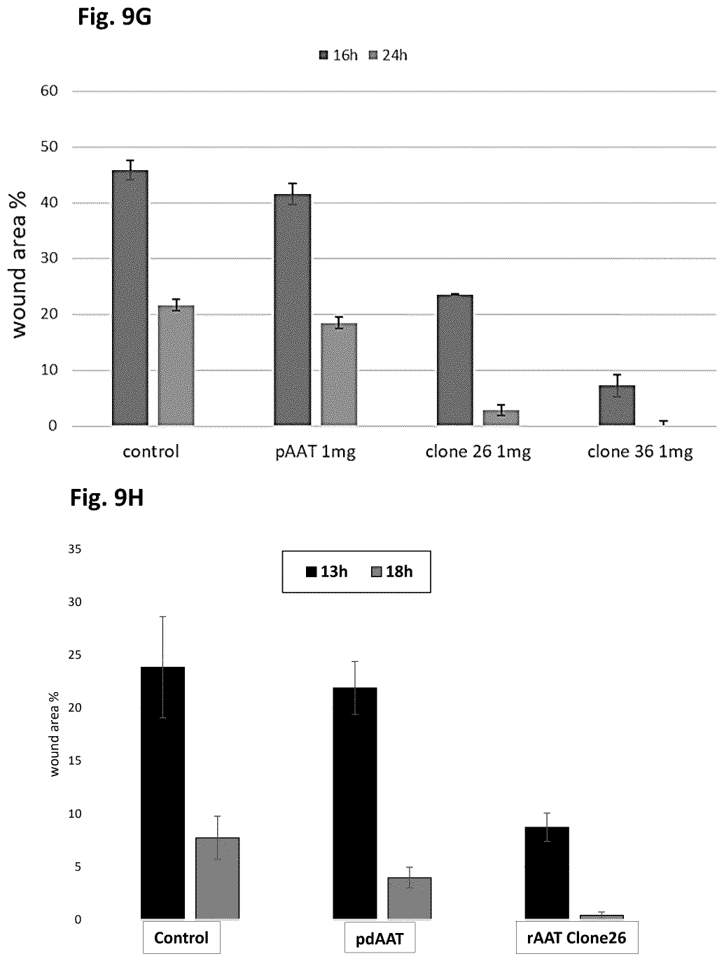

The results are presented in FIGS. 9A-G, which clearly demonstrate the enhanced wound healing effect of the rAATs tested, as compared to control treatment or the pdAAT.

### 2. Human Umbilical Vein Endothelial Cells

Cells were seeded in Ibidi culture-insert, two compartments with a defined gap, 21,000 cells on each side of the insert.
Mitomycin C was used to inhibit proliferation and distinguish between proliferation and migration of the cells.
Cells were treated with 1 mg/ml of pdAAT or rAAT in 5% serum medium.
Control was treated by vehicle (saline).
Pictures of the cells were taken at time 0, 13 and 18 hours. wound closure was calculated by ImageJ and presented as percentage of wound closure after 13 and 18 hours.

The results are presented in the graph shown in FIG. 9H, which clearly demonstrate that rAAT has an enhanced wound healing effect (as determined by percentage of wound closure) on endothelial cells, as compared to control and pdAAT.

### REFERENCES

1. Viglio, S., et al., Methods of Purification and Application Procedures of Alpha1 Antitrypsin: A Long-Lasting History. Molecules, 2020. 25(17): p. 4014.
2. Daemen, M. A., et al., Functional protection by acute phase proteins α1-acid glycoprotein and α1-antitrypsin against ischemia/reperfusion injury by preventing apoptosis and inflammation. Circulation, 2000. 102(12): p. 1420-1426.
3. Eltzschig, H. K. and T. Eckle, Ischemia and reperfusion—from mechanism to translation. Nat Med, 2011. 17(11): p. 1391-401.
4. Soares, R. O. S., et al., Ischemia/Reperfusion Injury Revisited: An Overview of the Latest Pharmacological Strategies. Int J Mol Sci, 2019. 20(20).
5. Kalogeris, T., et al., Cell biology of ischemia/reperfusion injury. Int Rev Cell Mol Biol, 2012. 298: p. 229-317.
6. Virani, S. S., et al., Heart Disease and Stroke Statistics-2020 Update: A Report From the American Heart Association. Circulation, 2020. 141(9): p. e139-e596.
7. Seropian, I. M., et al., Anti-inflammatory strategies for ventricular remodeling following ST-segment elevation acute myocardial infarction. J Am Coll Cardiol, 2014. 63(16): p. 1593-603.
8. Toldo, S., et al., Alpha-1 antitrypsin inhibits caspase-1 and protects from acute myocardial ischemia-reperfusion injury. J Mol Cell Cardiol, 2011. 51(2): p. 244-51.
9. Mauro, A. G., et al., A preclinical translational study of the cardioprotective effects of plasma-derived alpha-1 anti-trypsin in acute myocardial infarction. Journal of Cardiovascular Pharmacology, 2017. 69(5): p. 273-278.
10. Carden, D. L. and D. N. Granger, Pathophysiology of ischaemia-reperfusion injury. The Journal of Pathology, 2000. 190(3): p. 255-266.
11. Mallick, I. H., et al., Ischemia-reperfusion injury of the intestine and protective strategies against injury. Dig Dis Sci, 2004. 49(9): p. 1359-77.
12. Demirkan, A., B. Sava, and M. Melli, Endotoxin level in ischemia-reperfusion injury in rats: effect of glutamine pretreatment on endotoxin levels and gut morphology. Nutrition, 2010. 26(1): p. 106-11.
13. Beutler, B. and E. T. Rietschel, Innate immune sensing and its roots: the story of endotoxin. Nat Rev Immunol, 2003. 3(2): p. 169-76.
14. Swamy, M., et al., Epithelial decision makers: in search of the ‘epimmunome’. Nat Immunol, 2010. 11(8): p. 656-65.
15. Kartalija, M., et al., Patients with nontuberculous mycobacterial lung disease exhibit unique body and immune phenotypes. American journal of respiratory and critical care medicine, 2013. 187(2): p. 197-205.
16. Schuster, R., et al., Distinct anti-inflammatory properties of alpha1-antitrypsin and corticosteroids reveal unique underlying mechanisms of action. Cell Immunol, 2020. 356: p. 104177.
17. Collins, C. B., et al., Alpha-1-antitrypsin therapy ameliorates acute colitis and chronic murine ileitis. Inflamm Bowel Dis, 2013. 19(9): p. 1964-73.
18. Ikebe, N., et al., Protective effect of S-nitrosylated alpha(1)-protease inhibitor on hepatic ischemia-reperfusion injury. J Pharmacol Exp Ther, 2000. 295(3): p. 904-11.
19. Moldthan, H. L., et al., Alpha 1-antitrypsin therapy mitigated ischemic stroke damage in rats. J Stroke Cerebrovasc Dis, 2014. 23(5): p. e355-63.

1.-29. (canceled)

30. A recombinant human alpha1-antitrypsin (rhAAT), comprising at least about 75% fucosylated N-linked glycans.

31. The rhAAT according to claim 30, comprising at least about 80% fucosylated glycans.

32. The rhAAT according to claim 30, wherein over about 80-95% of the fucose units are core fucose.

33. The rhAAT according to claim 30, wherein about 60-80% of the fucosylated N-linked glycans are Di-antennary.

34. The rhAAT according to claim 30, wherein about 5%-25% of the fucosylated N-linked glycans are Tri-antennary.

35. The rhAAT according to claim 30, wherein about 5%-20% of the fucosylated N-linked glycans are Tetra-antennary.

36. The rhAAT according to claim 30, having an increased immunomodulation activity as compared to immunomodulation activity of a purified plasma derived AAT (pdAAT).

37. The rhAAT according to claim 36, having at least about 5% higher immunomodulation activity as compared to immunomodulation activity of a purified plasma derived AAT (pdAAT).

38. The rhAAT according to claim 36, wherein said immunomodulation activity is characterized in reduction in activity, expression and/or secretion level of one or more of: IL-1 beta, TNF alpha, IL-6, IL8, IL 18, MCP1/CCL2.

39. The rhAAT according to claim 36, wherein said immunomodulation activity is characterized in increasing activity, expression and/or secretion level of IL-10 and/or IL-1-receptor antagonist (IL-1Ra).

40. The rhAAT according to claim 30, produced or expressed in Chinese hamster ovary (CHO) cell line.

41. The rhAAT according to claim 40, wherein said CHO cell line is CHO DG44 cell line and/or wherein said CHO cell line further express \acute{α}-2,6-sialyltransferases

42. A method for preventing or treating an inflammatory condition in a subject in need thereof, the method comprising administering a therapeutically effective amount of the rhAAT according to claim 1 or a pharmaceutical composition comprising the same.

43. The method according to claim 42, wherein the inflammatory condition is a pulmonary disease selected from the group consisting of alpha-1 antitrypsin deficiency (AATD), small airway disease, chronic bronchitis, emphysema, chronic obstructive pulmonary disease (COPD), cystic fibrosis, bronchiectasis, asthma, pneumonia, parenchymatic and fibrotic lung diseases or disorders, interstitial pulmonary fibrosis, re-inflammation, acute respiratory distress syndrome (ARDS), and sarcoidosis.

44. The method according to claim 42, wherein the inflammatory condition is selected from the group consisting of graft-versus-host disease (GVHD), ischemia-reperfusion injury, ischemia/reperfusion injury following transplantation, acute myocardial infarction, acute kidney injury, rheumatoid arthritis, septic arthritis, psoriatic arthritis, ankylosing spondylitis, Wegener's disease, Crohn's disease, ulcerative colitis, psoriasis, type I diabetes, dermatitis, pneumonia, sepsis, wound healing, and systemic lupus erythematosus.

45. The method according to claim 42, wherein the inflammatory condition is ischemia-reperfusion injury.

46. The method according to claim 42, wherein the pharmaceutical composition is administered by injection.

47. The method according to claim 46, wherein the pharmaceutical composition is administered at a dose of from about 200 mg/kg to about 350 mg/kg.

48. The method according to claim 42, wherein the pharmaceutical composition is administered by inhalation.

49. The method according to claim 48, wherein the pharmaceutical composition is administered at a dose of from about 20 mg/dose to about 200 mg/dose.


SEQUENCE LISTING (9 sequences)

SEQ ID NO: 1 - DNA, 1266 bp, synthetic construct
  source 1..1266: mol_type = other DNA
atggatctgctgcacaagaacatgaagcacctgtggttctttctgctgctggtggccgctcctagatgggtgttgtctgaagatcctcagggcgacgccgctcagaaaaccgatacctctcaccacgaccaggatcaccccaccttcaacaagatcacccctaacctggccgagttcgccttcagcctgtacagacagctggcccaccagtccaactctaccaacatcttcttcagccccgtgtctatcgccaccgcctttgctatgctgtctctgggcaccaaggctgacacccacgatgagatcctggaaggcctgaacttcaacctgacagagatccccgaggctcagatccacgagggctttcaagagctgctgagaaccctgaaccagcctgactctcagctccagctgacaaccggcaacggcctgtttctgtctgagggcctgaagctggtggacaagttcctggaagatgtgaagaagctgtaccactccgaggccttcaccgtgaacttcggcgataccgaggaagccaagaagcagatcaacgactacgtggaaaagggcacccagggcaagatcgtggacctggtcaaagagctggacagagacaccgtgttcgccctggtcaactacatctttttcaaaggcaagtgggaacgccccttcgaagtgaaggacacagaggaagaggacttccacgtcgaccaagtgaccaccgtgaaggtgcccatgatgaagcggctgggcatgttcaacatccagcactgcaagaaactgtcctcttgggtgctgctgatgaagtacctgggcaacgctaccgctatcttcttcctgcctgatgagggcaagctgcagcacctggaaaacgagctgacccacgacatcatcaccaagttcttggagaacgaggaccggcggagcgcttctctgcatctgcctaagctgtctatcaccggcacctacgacctgaagtctgtgctgggacagctgggcatcacaaaggtgttctctaacggcgccgatctgtccggcgtgacagaagaagctcctctgaagctgtccaaggccgtgcataaggctgtgctgaccatcgatgagaagggaacagaggccgctggcgccatgtttctggaagctatccctatgagcatccctcctgaagtgaagttcaacaagcccttcgtgttcctgatgatcgagcagaacaccaagtctccactgttcatgggcaaagtggtcaaccccacacagaagtgatag

SEQ ID NO: 2 - protein, 420 aa, synthetic construct
MDLLHKNMKHLWFFLLLVAAPRWVLSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 10 aa, synthetic construct
EDPQGDAAQK

SEQ ID NO: 4 - protein, 11 aa, synthetic construct
FMGKVVNPTQK

SEQ ID NO: 5 - protein, 10 aa, synthetic construct
EDPQGDAAQK

SEQ ID NO: 6 - protein, 7 aa, synthetic construct
VVNPTQK

SEQ ID NO: 7 - protein, 11 aa, synthetic construct
RLGMFNIQHCK

SEQ ID NO: 8 - protein, 11 aa, synthetic construct
AIPMSIPPEVK

SEQ ID NO: 9 - protein, 12 aa, synthetic construct
PFVFLMIEQNTK